(19) United States
(12) Patent Application Publication
(10) Pub. No.: US 2022/0265818 A1
(43) Pub. Date: Aug. 25, 2022

(54) CHIMERIC ANTIGENS FOR TREATING VIRAL INFECTION

(71) Applicant: AKSHAYA BIO INC., Edmonton (CA)

(72) Inventors: George RAJAN, Edmonton (CA); Allan MA, Edmonton (CA); Yuenian SHI, Roslyn Heights, NY (US)

(73) Assignee: AKSHAYA BIO INC., Edmonton (CA)

(21) Appl. No.: 17/570,965

(22) Filed: Jan. 7, 2022

Related U.S. Application Data
(63) Continuation of application No. PCT/US2020/040272, filed on Jun. 30, 2020.
(60) Provisional application No. 62/871,891, filed on Jul. 9, 2019.

(51) Int. Cl. A61K 39/29; A61P 31/14; A61K 38/21; A61K 47/60; A61K 31/713; A61K 31/7068

(52) U.S. Cl. CPC A61K 39/292; A61P 31/14; A61K 38/212; A61K 47/60; A61K 31/713; A61K 31/7068; A61K 2039/545

(57) ABSTRACT

Chimeric antigens and use there of in combination with antiviral agents to elicit immune responses against chronic Hepatitis B infections, thus breaking tolerance to hepatitis B virus in the host.

### CROSS-REFERENCE TO RELATED APPLICATIONS

This application is a continuation of International Application No. PCT/US2020/040272, filed on Jun. 30, 2020, which claims priority to U.S. Patent Application No. 62/871,891, filed on Jul. 9, 2019. The disclosures of the aforementioned applications are hereby incorporated by reference in their entireties.

### REFERENCE TO A SEQUENCE LISTING VIA EFS-WEB

The content of the ASCII text file of the sequence listing (i. Name: “Sequence Listing”; ii. Date of Creation: May 12, 2022; and iii. Size: 2.2 kb) electronically submitted via EFS-Web on May 12, 2022, is incorporated herein by reference in its entirety.

### TECHNICAL FIELD

The present application relates to the use of chimeric antigens for breaking host tolerance to HBV antigens by targeting and activating antigen presenting cells (APCs) to induce HBV-specific T cell and antibody responses.

### BACKGROUND

Viral infectious diseases are major public healthcare issues. Human Hepatitis B virus (HBV) is a member of a family of DNA viruses that primarily infect the liver (Gust, 1986). Other members of this family are woodchuck hepatitis B virus (WHV, Summers et al., 1978), duck hepatitis B virus (DHBV, Mason et al., 1980) and heron hepatitis B virus (HHBV, Sprengel et al., 1988). These viruses share a common morphology and replication mechanisms, but are species-specific for infectivity (Marion, 1988).

HBV primarily infects liver cells and can cause acute and chronic liver disease resulting in cirrhosis and hepatocellular carcinoma. Infection occurs through blood and other body fluids. Approximately 90% of the adult individuals infected by HBV are able to clear the infection, while the remaining 10% become chronic carriers of the virus with a high probability of developing cirrhosis of the liver and hepatocellular carcinoma (Block, 2016). The World Health Organization statistics show that more than 2 billion people have been infected by HBV and among these, 350 million are chronically infected by the virus (Beasley, 1988; Lau and Wright, 1993). Prophylactic vaccines based on HBV surface antigen (HbsAg) have been very effective in providing protective immunity against HBV infections. These vaccines have been developed from HBsAg purified from plasma of chronic HBV carriers, produced by recombinant DNA techniques as well as through the use of synthetic peptides (Please see U.S. Pat. Nos. 4,599,230 and 4,599,231). These vaccines are highly effective in the prevention of infection, but are ineffective in eradicating established chronic infections.

Human Hepatitis B Virus (HBV) belongs to the family of Hepadnaviruses (Mason et al., 1980). Other members of this family are Duck Hepatitis B Virus (DHBV), Woodchuk Hepatitis

Virus (WHV), Ground squirrel Hepatitis B Virus (GSHV) and Heron Hepatitis B Virus (HHBV). Although these viruses have similar morphology and replication mechanism, they are fairly species specific consequently, infect only very closely related species. These viruses have a DNA genome ranging in size of 3.0-3.2 Kb, with overlapping reading frames to encode several proteins. HBV genome encodes several proteins. Among these, the surface antigens: Large (S1/S2/S), Medium

(S2/S) and Small (S) are proposed to be involved in the binding of the virus to the cellular receptors for uptake. The core protein (Core) forms capsids which encapsulate the partially double stranded DNA genome. Polymerase/Reverse Transcriptase (Pol) protein is a multifunctional enzyme necessary for the replication of the virus. The X protein has been proposed to have many properties, including the activation of Src kinases (Ganem and Schneider, 2001). The present application describes the use of chimeric antigen fusion protein of HBV S1 fragment/S2 fragment/Core protein and a murine (xenotypic) Mab Fc fragment in combination with other HBV antiviral agents.

DHBV, another member of the hepadnaviral family, infects Pekin ducks which results in chronic DHBV infection. This is species-specific, and has served as an animal model for studying the hepatitis B viruses. DHBV has a DNA genome and it codes for surface antigens PreS and PreS/S, Core protein (Core) and Polymerase/Reverse Transcriptase.

When a healthy host (human or animal) encounters an antigen (such as proteins derived from a bacterium, virus and/or parasite), normally, the host initiates an immune response. This immune response can be a humoral response and/or a cellular response. In the humoral response, antibodies are produced by B-cells and are secreted into the blood and/or lymph in response to an antigenic stimulus. The antibody then neutralizes the antigen, e.g. a virus, by binding specifically to antigens on its surface, marking it for destruction by phagocytotic cells and/or complement-mediated mechanisms. The cellular response is characterized by the selection and expansion of specific helper and cytotoxic T-lymphocytes capable of directly eliminating the cells which contain the antigen.

In many individuals, the immune system does not respond to certain antigens. When an antigen does not stimulate the production of a specific antibody and/or killer T-cells, the immune system is unable to prevent the resultant disease. As a result, the infectious agent, e.g. virus, can establish a chronic infection and the host immune system becomes tolerant to the antigens produced by the virus. The mechanism by which the virus evades the host immune machinery is not clearly established. The best-known examples of chronic viral infections include Hepatitis B, Hepatitis C, Human Immunodeficiency Virus and Herpes Simplex Virus.

In chronic states of viral infections, the virus escapes the host immune system. Viral antigens are recognized as “self”, and thus are not recognized by the antigen-presenting cells. Antigen Presenting Cells process the encountered antigens differently depending on the localization of the antigen (Steinman et al., 1999). Exogenous antigens are processed within the endosomes of the APC and the generated peptide fragments are presented on the surface of the cell complexed with Major Histocompatibility Complex (MHC) Class II. The presentation of this complex to CD4+ T cells stimulates the CD4+ T helper cells. As a result, cytokines secreted by the helper cells stimulate B cells to produce antibodies against the exogenous antigen (humoral response). Immunizations using antigens typically generate antibody response through this endosomal antigen processing pathway.

On the other hand, intracellular antigens are processed in the proteasome and the resulting peptide fragments are presented as complexes with MHC Class I on the surface of APCs. Following binding of this complex to the co-receptor CD8 molecule, antigen presentation to CD830 T cells occurs which result in cytotoxic T cell (CTL) immune response to remove the host cells that carry the antigen.

In patients with chronic viral infections, since the virus is actively replicating, viral antigens will be produced within the host cell. Secreted antigens are present in the circulation. As an example, in the case of chronic HBV carriers, virions, the HBV surface antigens and the core antigen (e-antigen) can be detected in the blood. An effective therapeutic vaccine may be able to induce CTL responses against an intracellular antigen or an antigen delivered into the appropriate cellular compartment so as to activate the MHC Class I processing pathway.

A therapeutic vaccine that can induce a CTL response may be processed through the proteasomal pathway and presented via the MHC Class I (Larsson et al., 2001). This can be achieved either by producing the antigen within the host cell, or can be delivered to the appropriate cellular compartment so that it gets processed and presented so as to elicit a cellular response. Several approaches have been documented in the literature for the intracellular delivery of the antigen. Among these, viral vectors (Lorenz et al., 1999), the use of cDNA-transfected cells (Donnelly et al., 1997) as well as the expression of the antigen through injected cDNA vectors (Lai and Bennett, 1998; U.S. Pat. No. 5,589,466), have been documented. Several other approaches to treat chronic HBV infection have been cited in the literature (Liang et al., 2015; Block et al., 2015; Block et al., 2018).

Delivery vehicles capable of carrying the antigens to the cytosolic compartment of the cell for MHC Class I pathway processing have also been used. The use of adjuvants to achieve the same goal has been described in detail (Hilgers et al., 1999). Another approach is the use of biodegradable microspheres in the cytoplasmic delivery of antigens (Newman et al., 2000), exemplified by the generation of a Th1 immune response against ovalbumin peptide (Newman et al., 2000; Newman et al., 1998). It has also been shown that PLGA nanospheres are taken up by the most potent antigen presenting cells, dendritic cells (Newman et al., 2002).

Dendritic cells (DCs) derived from blood monocytes, by virtue of their capability as professional antigen presenting cells may be immune modulators which stimulate primary T cell response (Steinman et al., 1999; Banchereau and Steinman, 1998). This unique property of the DCs to capture, process, present the antigen and stimulate naïve T cells has made them very important tools for therapeutic vaccine development (Laupeze et al., 1999). Targeting of the antigen to the DCs is an important step in the antigen presentation and the presence of several receptors on the DCs for the Fc region of monoclonal antibodies have been exploited for this purpose (Regnault et al., 1999). Examples of this approach include ovarian cancer Mab-B43.13, Anti-PSA antibody as well as Anti-HBV antibody antigen complexes (Wen et al., 1999). Cancer immunotherapy using DCs loaded with tumor associated antigens have been shown to produce tumor-specific immune responses and anti-tumor activity (Campton et al., 2000; Fong and Engleman, 2000). Promising results were obtained in clinical trials in vivo using tumor-antigen-pulsed DCs (Tarte and Klein, 1999). These studies clearly demonstrate the efficacy of using DCs to generate immune responses against cancer antigens.

In recent years, active immunotherapy for cancer has been used for treating cancer. A major component of this approach is the blockage of programmed cell death using PD-1 and PD-L1 also known as “Check Point Inhibitors”. Commercially available products include Pembrolizumab (Merck) for treating melanoma and non-small cell lung cancer. Similarly, Nivolumab (Bristol-Myers Squibb) is approved for treating melanoma, squamous cell lung cancer, renal cell carcinoma and Hodgkin's lymphoma. Similar approach to treat chronic HBV infections have been researched and reported in the literature (Peng et al., 2008). In a mouse model for persistent HBV infection, PD-1 blockage has been shown to reverse some of the immune dysfunction (Tzeng, H. T et al., 2012; Fisicaro et al., 2010)

Chimeric antigens (fusion of Fc antibody fragment and HBV antigens) have been shown to be effective for eliciting HBV-antigen specific T and B cell mediated immune responses against HBV and also break tolerance to HBV antigens in chronic conditions (George et. al., U.S. Pat. Nos. 8,007,805 B2, 8,025,873 B8,029,803 B2, 8,465,745 B2, China patents 200480022747.1, India 225281, Japan 5200201, Korea 10-1327719, New Zealand 545048, Singapore 119567, Taiwan 1364294, South Africa 2006-0804, Australia 2012200998, European Patent validated; Germany, France, UK 1664270; Ma et al., 2020, George et al., 2020).

### SUMMARY

In one aspect, the application is directed to a method of treating viral infection in a host by administering a chimeric antigen and a therapeutic agent that decreases viral levels in the host.

In some embodiments of the method, the chimeric antigen is HBV S1/S2/Core chimeric antigen.

In other embodiments of the method, the therapeutic agent is an HBV antiviral agent. In some examples, the HBV antiviral agent may be a pharmaceutically acceptable agent comprising one or more compounds selected from the group consisting of: tenofovir, tenofovir disoproxil fumarate, tenofovir alafenamide, entecavir, telbuvidine, adefovir dipivoxil, and lamivudine. In other examples, the HBV antiviral agent is an HBV antiviral RNAi agent that inhibits HBV replication. In still other examples, the method of claim 3, wherein the HBV antiviral agent is a core capsid assembly inhibitor. In still further examples, the method of claim 3, wherein the HBV antiviral agent is a TLR agonist.

In still other embodiments of the method, the method further comprises administering an immune modulatory agent to the host. In some examples, the immune modulatory agent is an interferon such as Peginterferon α2-a. In other examples, the immune modulatory agent is an immune checkpoint inhibitor such as PD1/PDL1 inhibitor.

In still further embodiments of the method, the host is a human subject. In other embodiments, the viral infection is an HBV infection.

In another aspect, the application is directed to a kit for treating viral infection in a host, comprising a chimeric antigen and a therapeutic agent that decreases viral levels in the host, and a package insert or label with directions to treat viral infection in the host by administering to the host the therapeutic chimeric antigen and a therapeutic agent.

In some embodiment of the kit, the directions comprise instructions to administer the chimeric agent and the therapeutics agent simultaneously. In some examples, the directions comprise instructions to administer the chimeric agent and the therapeutic agent separately.

In some embodiments of the kit, the chimeric antigen is HBV S1/S2/Core chimeric antigen. In other embodiments, the therapeutic agent is an HBV antiviral agent. In some examples, the HBV antiviral agent is a pharmaceutically acceptable agent comprising one or more compounds selected from the group consisting of: tenofovir, tenofovir disoproxil fumarate, tenofovir alafenamide, entecavir, telbuvidine, adefovir dipivoxil, and lamivudine. In other examples, the HBV antiviral agent is an HBV antiviral RNAi agent that inhibits HBV replication. In still other examples, the HBV antiviral agent is a core capsid assembly inhibitor. In further examples, the HBV antiviral agent is a TLR agonist.

In other embodiments, the kit further comprises an immune modulatory agent. In some examples, the immune modulatory agent is an interferon such as Peginterferon α2-a. in other examples, the immune modulatory agent is an immune checkpoint inhibitor such as PD1/PDL1 inhibitor.

In a further aspect, the present application is directed to use of a chimeric antigen and a therapeutic agent that decreases viral levels in the host in treating viral infection in a host. In some embodiments, the chimeric antigen is HBV S1/S2/Core chimeric antigen. In some embodiments, the therapeutic agent is an HBV antiviral agent. In some examples, the HBV antiviral agent is a pharmaceutically acceptable agent comprising one or more compounds selected from the group consisting of: tenofovir, tenofovir disoproxil fumarate, tenofovir alafenamide, entecavir, telbuvidine, adefovir dipivoxil, and lamivudine. In other examples, the HBV antiviral agent is an HBV antiviral RNAi agent that inhibits HBV replication. In still other examples, the HBV antiviral agent is a core capsid assembly inhibitor. In further examples, the HBV antiviral agent is a TLR agonist.

In other embodiments, the use further comprises an immune modulatory agent. In some examples, the immune modulatory agent is an interferon such as Peginterferon α2-a. In other examples, the immune modulatory agent is an immune checkpoint inhibitor such as PD1/PDL1 inhibitor.

### BRIEF DESCRIPTION OF THE DRAWINGS

The patent or application file contains at least one drawing executed in color. Copies of this patent or patent application publication with color drawings will be provided by the Office upon request and payment of the necessary tee.

FIG. 1 shows a schematic diagram illustrating the structure of the chimeric antigen in its assembled state as a dimer. The chimeric antigen comprises HBV S1 and S2 fragments, Core antigen and a murine monoclonal antibody Fe fragment. (N-terminus) bel lis rTEV Protease Site-HBV S1/S2/Core [IRD] - - - Linker Peptide-Part CH1-CH2-C3-Peptide [TBD] (C-terminus).

FIG. 2 shows a schematic representation of the DHBV Core Chimeric antigen Vaccine in its dimerized form. (N-terminus) 6xHis-rTEV Protease Site-DHBV Core [IRD] - - - Linker Peptide-Part CH1-CH2-CH3-Peptide [TBD] (C-terminus).

FIG. 3 shows a chart depicting events in recombination.

FIG. 4 shows two graphs depicting Binding of DHBV core chimeric antigen and TBD to duck PBMC, shown as % positive cells (Left Panel) and Relative Mean Fluorescence Intensity (Right Panel).

FIG. 5 shows a graph depicting inhibition of the binding of DHBV core Chimeric antigen to duck PBMC by Fc fragments.

FIG. 6 shows a photo depicting DHBV DNA in Post-Hatch DHBV-Infected Ducks.

FIGS. 7A and 7B show graphs depicting Anti-DHBV Core Antibody Levels in Persistently Infected Duck Sera that are immunized with PBS (7A) or with core-TBD (7B), respectively.

FIGS. 8A and 8B show photos depicting DHBV DNA in Persistently DHBV-infected Duck Sera Following 3TC and vaccine Administration, respectively.

FIG. 9 shows a graph depicting Mean SGPT (ALT) levels in persistently infected ducks.

FIG. 10 shows a graph depicting Mean SGOT (AST) levels in persistently infected ducks.

FIG. 11 shows a schematic representation of HBV core chimeric antigen vaccine in a dimerized form.

FIGS. 12A and 12B show graphs depicting effect of HBV chimeric vaccine on liver

HBV DNA in transgenic mice using qPCR (FIG. 12A) and Southern blot hybridization (FIG. 12B).

FIGS. 13A and 13B show graphs depicting effect of HBV Core Chimeric antigen vaccine on Plasma ALT (FIG. 13A) and liver HBV RNA in transgenic mice (FIG. 13B).

FIG. 14 shows a graph depicting effect of HBV Core chimeric antigen vaccine on whole body weight change in transgenic mice.

FIGS. 15A-15P show graphs depicting effect of HBV Core chimeric antigen vaccine on liver cytokines (IL-1α, IL-1β, IFN-γ, IL-2, IL-3, IL-4, IL-5, IL-6, IL-9, IL-10, IL-12, MCP-1, TNF-α, MIP-1, GM-CSF, and RANTES, respectively, in HBV transgenic mice.

FIGS. 16A-16P show graphs depicting effect of HBV Core chimeric antigen vaccine on plasma cytokines (IL-1α, IL-1β, IFN-γ, IL-2, IL-3, IL-4, IL-5, IL-6, IL-9, IL-10, IL-12, MCP-1, TNF-α, MIP-1, GM-CSF, and RANTES, respectively, in HBV transgenic mice.

FIGS. 17A-17P show graphs depicting effect of HBV Core chimeric antigen vaccine on spleen cytokines (IL-1α, IL-1β, IFN-γ, IL-2, IL-3, IL-4, IL-5, IL-6, IL-9, IL-10, IL-12, MCP-1, TNF-α, MIP-1, GM-CSF, and RANTES, respectively, in HBV transgenic mice.

FIGS. 18A-18D are graphs depicting temporal expression of plasma IFN-γ in response to HBV Core chimeric antigen vaccine in HBV transgenic mice for all values at day 1, 14, 35 and 56, respectively.

FIG. 19 shows a graph depicting temporal expression of plasma IFN-γ in response to HBV Core chimeric antigen vaccine in HBV transgenic mice for combined values of all animals treated with HBV Core chimeric antigen vaccine.

### DETAILED DESCRIPTION OF THE EMBODIMENTS

The present application is based on the finding that chimeric antigens, in combination with anti-HBV antiviral agents and/or immune modulatory agents can break tolerance to HBV antigens by inducing HBV-specific T and B cell responses in HBV carrier which may result in the clearance of chronic HBV infection. Specifically, the chimeric antigens are molecules that couple viral antigens, such as Hepatitis B core, to a xenotypic fragment, such as a murine immunoglobulin G Fc fragment.

In some aspects, the present application describes the use of chimeric antigens in combination with one or more of antiviral agents for decreasing the viral load in hosts and also the use of stimulatory/inhibitory immunology agents to augment the immune response by chimeric antigens. In some embodiments, the chimeric antigens use DHBV Core protein fusion with a fragment of a xenotypic mAb, which may elicit a broad immune response in chronic viral infections, thus as therapeutic vaccine.

Examples of antiviral agents include, but not limited to, Nucleoside/Nucleotide analogues RNAi, Capsid assembly inhibitors, TLR agonists, Interferons, Immune Check point inhibitors, and any other agents which directly or indirectly decrease the viremia.

Examples of immune modulatory agents include but not limited to, Immune Check point inhibitors, e.g., blocking antibodies for PD-1, PDL-1, and CTLA4, and any other agents which directly or indirectly modulate immune response to virus infection.

In chronic HBV infection treatment, the presentation of HBV antigens to elicit a CTL response by the use of vaccine molecules designed to target the vaccines to DCs whereby the HBV-associated antigens treated as “self” during the chronic infection will be recognized as “foreign” and the host's immune system will mount a CTL response to eliminate HBV-infected cells. At the same time, through cross presentation, the antibody response to the circulating HBV antigen will bind to the antigen and remove it from the circulation. Accordingly, the present application details therapeutic use of the chimeric antigens in combination with other antiviral agents to induce immune response in patients who have chronic HBV infection thus clear the infection. An exemplary chimeric antigen is shown in FIG. 1.

### EXAMPLES

### EXAMPLE 1

### Evaluation of Chimeric DHBV Antigen as Immunotherapy for Chronic DHBV Infection

1 Introduction

1.1 Animal Models of HBV

Human HBV belongs to the family of Hepadnaviruses. Other members of this family are the Duck Hepatitis B Virus (DHBV, Mason et al., 1980), the Heron Hepatitis B Virus (HHBV, Sprengel et al., 1988), the Woodchuck Hepatitis Virus (WHV, Summers et al., 1978) and the Ground squirrel Hepatitis B Virus (GSHV, Marion et al., 1980). Although these viruses share a very similar organization of the genome and the characteristics of their replication mechanism, they are fairly species specific and consequently infect only very closely related species. Pekin ducks (Anas platyrhynchos) infected with duck hepatitis B virus (DHBV) have served as an animal model for the study of the mechanism of viral replication and the screening for antiviral agents (Schultz et al., 2004). These viruses have a DNA genome ranging in size from 3.0 to 3.2 kb with overlapping reading frames encoding several proteins.

The HBV genome encodes several proteins. Among these, the surface antigens, Large (S1/S2/S), Medium (S2/S) and Small (S) are believed to be involved in the binding of the virus to the cellular receptors for internalization. The core protein (Core) forms capsids which encapsulate the partially double stranded DNA genome. Polymerase/Reverse Transcriptase (Pol) protein is a multifunctional enzyme necessary for the replication of the virus. The X protein is thought to have many functions (Ganem, 2001, Bouchard and Schneider, 2004), including the activation of Src kinases (Klein and Schneider, 1997).

The DHBV genome encodes the surface antigen PreS/S, the core protein and the polymerase/reverse transcriptase protein. Although DHBV does not contain an open reading frame for the X protein, it has been shown that there is an X-like protein produced in cultured cells (Chang et al., 2001), but the functional significance of this gene product in DHBV infection of ducks is unclear (Meier et al., 2003). Although HBV and DHBV show similarities, there are differences between these viruses in several aspects. DHBV is not pathogenic to the host. DHBV may lack the X gene product and it has been suggested that the absence of the development of hepatocellular carcinoma may result from the lack of this protein. The glycoprotein, carboxypeptidase D, critical for DHBV infection has not been observed in HBV (Schultz et al., 2004).

1.2 DHBV as Animal Model for HBV Infection

Ever since the discovery of DHBV (Mason et al., 1980) the duck animal model has been used extensively for studying the replication mechanism of hepadnaviruses and for antiviral drug screening (Schultz et al., 2004). In spite of the limited knowledge of the immune system of the ducks, this animal model has been used for studying the immune responses to DHBV infection (Jilbert and Kotlarski, 2000).

Similar to HBV infection in humans, DHBV infection in ducks can be either acute or chronic. The transmission of the infection can occur through the bloodstream of infected laying ducks into the eggs. Congenital DHBV infection occurs as a result of viral replication in the yolk sac and the developing liver of the embryo (Urban et al., 1985, Jilbert et al., 1998). Congenitally DHBV-infected ducks remain chronically infected for life, but with very minimal histological changes to the liver and no biochemical evidence of liver damage (Marion et al., 1984).

Persistent DHBV infection can also be produced in newly hatched ducklings by inoculation with infectious duck sera (Marion et al., 1984, Jilbert et al., 1996). The outcome of the infection seems to depend on the age of the duck with the most efficient infection occurring in the newly hatched ducklings and the efficiency going down with the increasing age of the birds (Jilbert et al., 1998). DHBV-infection in adult duck is transient, resulting in the production of neutralizing antibodies and immunity to re-infection (Jilbert et al., 1998). The type of immune responses resulting in the infection in the neonates and the lack of infection in the older birds are not clearly understood. It is suggested that the relative maturity of the immune system defines the outcome of the infection; the more developed the immune system, the higher the chance of virus clearance and the resultant immunity (Zinkernagel, 1996, Vickery and Cossart, 1996).

Congenitally and persistently DHBV-infected ducks as well as the primary hepatocytes derived from them have been used extensively to screen antiviral compounds and to study the mechanism of replication of the virus (Offensperger et al., 1993, 1996; Hafkemeyer et al., 1996).

1.3 Duck as Animal Model for the Study of Protective and Therapeutic Immunity

The duck animal model has also been used for the evaluation of prophylactic as well as therapeutic immune responses against DHBV (Rollier et al., 1999). DNA-based vaccines have been shown to have protective and therapeutic effect (Rollier et al., 2000a & b). The results arising from studies using antiviral compounds such as adefovir and DHBV L protein DNA-based vaccines suggest that a combination of antiviral drugs with immunotherapy may be more effective that either one alone for the treatment of chronic HBV infection (LeGuerhier et al., 2003).

1.4 Normal Immune Responses to HBV Infection

In order to achieve the elimination of intracellular pathogens such as HBV, it is generally believed that the infected cells have to be destroyed by MHC Class I-restricted CD8+ CTLs (Chisari and Ferrari, 1995). During HBV infection, the CTL responses are believed to cause liver cell inflammation and virus clearance (Bertoletti and Maini, 2000; Rehermann, 2000). The role of pro-inflammatory cytokines such as IFN-γ in the elimination of HBV without the killing of infected cells has also been documented (Guidotti et al., 1999; Bertoletti et al., 1997). This effect has been shown clearly in acutely HBV-infected chimpanzees (Guidotti et al., 1999). In the HBV transgenic mice, the induction of IFN-γ and TNF-α in the liver was able to abolish the replication and gene expression of the virus non-cytopathically (Guidotti et al., 1996). The antiviral effect of the inflammatory cytokines has been demonstrated in the HBV transgenic mice superinfected with LCMV. This process is believed to be mediated by TNF-α and IFN-α/β produced by the LCMV-infected hepatic macrophages (Guidotti et al., 1996a). These results emphasize the importance of immune responses required for the elimination of chronic HBV infections.

1.5 Duck Immune Responses to DHBV Infection

Studies on the immune responses to DHBV infection in the duck animal model have been very limited by the poor understanding of the duck immune system and the lack of appropriate reagents (Schultz et al., 2004). Recently, several duck cytokines have been identified. Among these, Duck IFN-γ has been shown to inhibit the replication of DHBV both in vitro and in vivo (Heuss et al., 1998, Schultz et al., 1999).Very limited information about other duck cytokines is available although some studies have been reported about IFN-γ (Schultz and Chisari, 1999) and IL-2 (Schultz et al., 2004). In spite of the lack of availability of immunological tools for the evaluation of the immune responses in the duck animal model, we have attempted to use this model to study the effect of chimeric antigen vaccines.

1.6 Design of DHBV Core Chimeric Antigen Vaccine

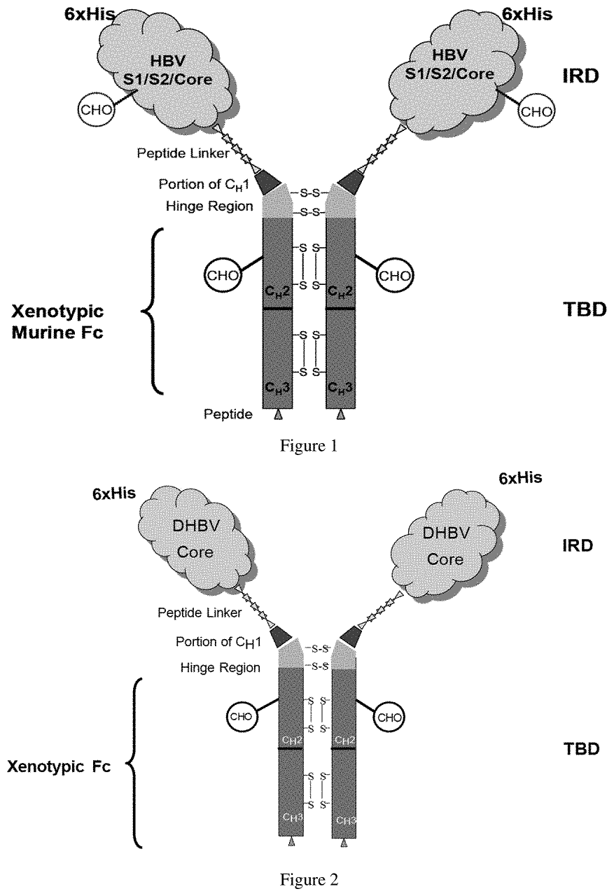

Chimeric antigen vaccine technology represents a breakthrough in the design and development of unique anti-viral therapeutic vaccines. Chimeric antigen vaccines are a novel class of recombinant “chimeric antigens” which contain selected antigens and specific regions of a xenotypic antibody, produced as fusion proteins. The bifunctional design of the molecule is tailored to target the viral antigen to the proper antigen presenting cells in order to break the tolerance to the viral antigen and to elicit both humoral and the most desirable cellular immune response to clear the viral infection. Chimeric antigen vaccine monomeric fusion protein can be schematically represented in FIG. 2.

As shown in FIG. 2, the vaccine has two domains; an immune response domain (IRD) that contains the recombinant antigen and a target-binding domain (TBD) which contains a xenotypic antibody fragment portion. More specifically, the chimeric antigen contains DHBV Core antigen fused to a Xenotypic (murine) Fc fragment.

The design of the molecule imparts several unique properties to its function. The unique chimeric design favors the formation of antibody-like structures that facilitate its uptake through specific receptors and results in appropriate antigen presentation. It can generate a broad immune response, both cellular (class I) and humoral (class II). It can be processed through the proteasomal pathway and the peptides presented as complexes with MHC class I, resulting in a CTL response. Chimeric antigen vaccines can also be processed via the endosomal pathway, presented by MHC class II, and produce a humoral response. The TBD mediates the binding of the Chimeric antigen vaccine to specific APC receptors such as Fc-y receptors. Binding to immature DCs predominantly results in class I presentation. The xenotypic nature of the TBD makes the molecule more immunogenic. In addition, the linker peptides of varying lengths, unrelated to the native proteins, incorporated at the N and C termini of the antigen and the antibody fragments in the Chimeric antigen vaccine help the molecule to be recognized as “foreign” by the host immune system of a chronic DHBV carrier.

The expression of the recombinant proteins in insect cells imparts glycosylation different from mammalian, a feature that adds another dimension to the immunogenicity of the protein. Mannose/pauci mannose glycosylation introduced in insect cells also permits the targeting of mannose receptors on APCs for uptake. Chimeric antigen vaccines can be taken up by the APCs through specific Fcγ receptors I, II and III (CD64, CD32, CD16), mannose receptors (CD206), other C-type lectin receptors and through phagocytosis (Geijtenbeek et al., 2004). The uptake via these receptors, processing through the endosomal and proteasomal pathways and presentation on both classes of MHCs can result in a broad immune response to eliminate the virus-infected cells and remove any circulating antigen.

Generation of a CTL response is critical to clear virus-infected cells. In antibody-based therapies, the administered antibody concentration has to be adjusted to each patient for the optimal complexing of the circulating autologous antigen. In the Chimeric antigen vaccine technology, this variable is avoided since the stoichiometry of Ag/Ab is maintained in the vaccine molecule. An added advantage is that DHBV Chimeric antigen vaccines do not have to rely on circulating antigen for the presentation.

2. Generation of DHBV Core Chimeric Antigen Vaccine

2.1 Cloning and Expression

The following sections describe the methodology used for the cloning of the DHBV Core Chimeric antigen vaccine, DHBV core and TBD component using the “Bac to Bac” cloning system. This section describes the standardization of the expression of the proteins in High Five™ insect cells.

2.2 pFastBacHTa TBD, the Parent Plasmid Construct

The mouse IgG1 DNA sequences encoding amino acids of CH1-Hinge-CH2-CH3 reg ion were generated from mRNA isolated from the hybridoma (2C12) which produces mAb again st HBsAg. Total mRNA was isolated using TRizol reagent (Thermo Fisher Scientific) and the cD NA of the TBD was generated by RT-PCR using Superscript First-strand Synthesis (Thermo Fish er Scientific). The PCR primers contained linker sequences encoding the linker peptide—SRPQG GGS—at the 5′ terminus, a unique Not I site at the 5′ and a unique Hind III restriction site at the 3′ end. The resulting cDNA contains (5′ Not I)-linker sequence-CH1 (VDKKI)-CH2-CH3-Linker sequence-(3′ Hind III). Following digestion with the respective enzymes, the fragment was ligate d with pFastBacHTa donor vector plasmid (Thermo Fisher Scientific) using the same restriction enzyme sites. The 5′ primer used for PCR amplification was (Sense) 5′ TGTCATTCTGCGGCC G CAAGGCGGCGGATCCGTGGACAAGAAAATTGTGCCCAGG (SEQ ID NO: 1)and the 3′ primer used was (antisense) 5′ ACGAATCAAGCTTTGCAGCCC AGGAGAGTGGGAGAG (SEQ ID NO: 2) which contained the Not I and Hind III sites, respectively.

The amplified DNA was digested with Not I and Hind III, the fragment purified by agarose gels and ligated with pFastBacHTa vector plasmid digested with the same restriction enzymes, to produce the donor plasmid pFastBacHTa-TBD. This plasmid was used for the transformation of E. coli DH10Bac to generate the recombinant Bacmids as described later. The DNA sequence and the accuracy of the ORF were verified by standard sequencing methods.

2.3 pFastBacHTa DHBV Core Antigen and DHBV Core-TBD Fusion Protein Expression Vectors

The DNA encoding the DHBV core was generated from the plasmid pRSET B DHBV Core using PCR methodology. The primers used were: (sense) 5′ TGCGCTACCATGGATATCAATGCTTCTAGAGCC (SEQ ID NO: 3) containing the unique restriction enzyme site Nco I and the 3′ primer (antisense) 5′ TGTCATTCTGCGGCCGCGATTTCCTAGGCGAGGGAGATCTATG (SEQ ID NO: 4) containing the unique restriction enzyme site Not I. Amplified DNA was digested with Nco I/Not I and ligated with pFastBacHTa expression vector digested with the same two enzymes. This produced the expression vector pFastBacHTa DHBV Core. This plasmid was used for the transposition in DH10Bac for producing the recombinant bacmid. The recombinant Bacmid was used for Sf9 insect cell transfections. The resulting baculovirus carrying the gene of DHBV Core was used for protein DHBV Core protein expression. The generation of pFastBacHTa DHBV Core-TBD plasmid was achieved through similar protocols. The amplified DNA by PCR was digested with Nco I/Not I and the purified DNA fragment was ligated with the plasmid pFastBacHTa-TBD following the digestion with the respective enzymes. This produced the expression vector pFastBacHTa DHBV Core-TBD. This plasmid was used to produce recombinant baculovirus which expressed the protein chimeric antigen DHBV Core-TBD.

2.4 Transformation of E. coli. DH5α

Ligated plasmids were used to transform E. coli DH5α and the plasmids were isolated by standard protocols. Sequence and open reading frames were verified by sequencing and the plasmids were used for the transformation of E. coli DH10Bac to generate the recombinant Bacmids.

2.5 Transposition

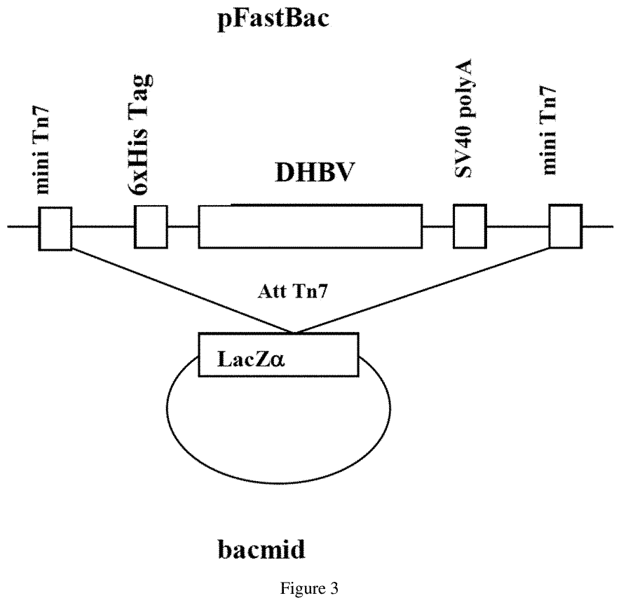

The generation of recombinant bacmids is based on the “Bac-To-Bac” cloning system (Thermo Fisher Scientific) that uses site-specific transposition with the bacterial transposon Tn7. This is accomplished in E. coli strain DH10Bac. The DH10Bac cells contain the bacmid, which confers kanamycin resistance and a helper plasmid which encodes the transposase and confers resistance to tetracycline. The gene of interest is cloned into the donor plasmid pFastBacHTa which has mini-Tn7 elements flanking the cloning sites. The plasmid is used to transform E. coli strain DH10Bac, which has a baculovirus shuttle plasmid (bacmid) containing the attachment site of Tn7 within a LacZa gene. Transposition disrupts the LacZα gene so that only recombinants produce white colonies and are easily selected for. The advantage of using transposition in E. coli is that single colonies contain only the recombinant bacmid. The recombinant bacmids are isolated using standard plasmid isolation protocols and are used for transfection in Sf9 insect cells to generate baculoviruses that express recombinant proteins. The sequence of events involved in the recombination is shown in FIG. 3.

Donor plasmids pFastBacHTa-TBD, pFastBacHTa DHBV Core, and pFastBacHTa DHBV Core-TBD were used for the site-specific transposition of the cloned gene into a baculovirus shuttle vector (Bacmid). The recombinant pFastBacHTa plasmids with the gene of interest (TBD, DHBV Core & Core-TBD) were used to transform DH10Bac cells for the transposition to generate recombinant bacmids.

A 100 μl aliquot of competent DH10Bac cells was thawed on ice, the pFastBacHTa based plasmids were added and the mixture was incubated on ice for 30 minutes. The mixture was given a heat shock for 45 seconds at 42° C. and then chilled on ice for 2 minutes. The mixture was then added to 900 μl of LB media and incubated for 4 hours at 37° C. The transformed cells are serially diluted with LB to 10-1 and 10-2 and 100 μl of each dilution is plated on LB agar plates supplemented with 50 μg/mL kanamycin, 7 μg/mL gentamicin, 10 μg/mL tetracycline, 100 μg/mL X-gal, and 40 μg/mL IPTG and incubated for at least 36 hours at 37° C.

The gentamicin resistance was conferred by the pFastBacHTa and the X-gal and IPTG were used to differentiate between white colonies (recombinant bacmids) from blue colonies (non-recombinant). The white colonies were picked and inoculated into 2 mL of LB supplemented with 50 μg/mL kanamycin, 7 μg/mL gentamicin and 10 μg/mL tetracycline and incubated overnight at 37° C. with shaking. A sterile loop was used to sample a small amount of the overnight culture and the sample was streaked onto a fresh LB agar plate supplemented with 50 μg/mL kanamycin, 7 μg/mL gentamicin, 10 μg/mL tetracycline, 100 μg/mL X-gal, and 40 μg/mL IPTG and incubated for at least 36 hr at 37 ° C. to confirm a white phenotype.

Recombinant bacmids were isolated by standard protocols (Sambrook & Russell, 2001), the DNA sample was dissolved in 40 μl of TE (10 mM Tris-HCL pH 8, 1 mM EDTA) and used for transfections.

2.6 Transfection and Production of Recombinant Baculovirus

In order to produce recombinant baculoviruses, the individual bacmid was transfected into Sf9 insect cells. Sf9 cells (9×105) were seeded into each well of a 6 well cell culture dish (35 mm wells) in 2 mL Sf 90011 (Thermo Fisher Scientific) and allowed to attach for at least 1 hr at 27° C. Transfections were carried out using Cellfectin Reagent (Thermo Fisher Scientific) as per the protocols provided by the supplier of the Sf9 cells. Following transfection, the cells were incubated at 27° C. for 72 hr. The medium containing baculovirus was collected and stored at 4° C. in the dark.

The efficiency of the transfection was verified by checking for production of baculovirus DNA. The isolated baculovirus DNA was subjected to PCR to screen for the inserted gene of interest. The expression of the heterologous protein in the cells was verified by SDS polyacrylamide gel electrophoresis (SDS-PAGE) and Western blots using the 6xHis tag monoclonal antibody (Clontech) as the probe.

2.7 Amplification of the Recombinant Baculovirus Stock

Once the production of the baculovirus and the expression of protein have been confirmed, the virus production is amplified to produce a concentrated stock of the baculovirus that carries the gene of interest. It is standard practice to amplify the baculovirus at least two times, and in all protocols described herein this standard practice was followed. After the second round of amplification, the concentration of the generated baculovirus was quantified using a plaque assay according to the protocols described by the manufacturer of the kit (Thermo Fisher Scientific). The most appropriate concentration of the virus to infect Sf9 insect cells and the optimum time point for the production of the desired protein were established as well. The protocols for the expression for both monolayer as well as suspension culture of Sf9 cells were established.

2.8 Expression of the Recombinant Proteins

Recombinant baculovirus of standardized MOI were used to infect High Five™ insect cells. For suspension cultures, cells were seeded at a density of 3×10⁵ cells/mL and incubated at 27.5° C. with shaking at 138 rpm until the cell density reached 2-3×10⁶ cells/mL Recombinant baculovirus was added to the cells. For the expression of DHBV proteins, the MOI was 1-2 pfu/cell and for TBD 10 pfu/cell was used. The incubation at 27.5° C. was continued for 48 hr. The cells were harvested by centrifugation at 2500 rpm for 10 minutes at 4 ° C. and used for the purification of the recombinant proteins. Unused portions of cells are snap frozen in liquid nitrogen and stored at −70° C.

2.9 Purification of Proteins

Purification of the 6xHis-tagged proteins is based on Ni-chelation affinity chromatography under denaturing conditions, followed by ion exchange chromatography. The protocols used for the purification of different proteins are described in the following sections.

2.10 Lysis and Solubilization

The cell pellet from 250 mL culture was resuspended in approximately 30 mL of ice-cold Lysis Buffer (6 M Guanidine HCl, 100 mM NaH2PO4, 10 mM Tris HCl, 10 mM Imidazole, pH 8.00). Following cooling on ice for 10 min, the suspension was subjected to sonication (Sonicator 3000, Misonics Inc., 60-65 Watts 1 min. pulse five times with 1-5 min. cooling in between). The lysate was solubilized further by stirring at RT for 2 hr. Unsolubilized material was removed by centrifugation in a JA 25.50 rotor at 15,000 rpm for 30 minutes, at 4° C. in a Beckman Avanti J25 centrifuge.

2.11 Ni-NTA Purification

BioRad Econo column (1.5×12 cm) was packed with 2.5 ml (PV) Ni-NTA Super Flow beads (Qiagen) and equilibrated with 20 mL of lysis buffer. The cleared lysate was loaded onto the Ni-NTA Super Flow column in order to bind the target protein.

The column was washed with lysis buffer (about 30 column volumes) until the A280 is stable. The column was washed further with approximately 30 column volumes of wash buffer (8 M urea in 100 mM NaH₂PO₄, 10 mM Tris, 20 mM Imidazole, pH 8.0) until the A280 was less than 0.1.The proteins bound to the Ni-NTA were eluted with elution buffer (8 M urea in 100 mM NaH₂PO₄, 10 mM Tris, 250 mM Imidazole, pH 8.0), 1 ml fractions were collected. Protein fractions with absorbance A280 (Ultrospec 4000 Spectrophotometer, Pharmacia Biotech) higher than 0.05 were pooled, concentrated using Amicon Ultra Centifuge Filter Devices (Millipore Amicon Ultra-15, 30,000 MWCO), into a final volume of 4-5 mL. The sample was applied to a Sephadex G 25 (1.5×30 cm) column pre-equilibrated with 8 M urea containing 20 mM NaH₂PO₄ pH 7.4, in order to remove the imidazole. The bound proteins were eluted from the column with the same buffer and the fractions containing the protein of interest were pooled and used for ion exchange chromatography.

2.12 Ion Exchange Chromatography using CM-Sepharose (DHBV Core Chimeric antigen vaccine and DHBV Core)

This procedure was used as a second step purification for DHBV Core Chimeric antigen vaccine and DHBV Core protein. CM Sepharose (Pharmacia) column (1.5×15 cm) was packed and equilibrated with 20 mM sodium phosphate, 8 M urea, pH 7.4. The proteins purified by Ni-NTA affinity chromatography were applied to the column. The flow rate used for binding and subsequent elution is 1 ml/min. The column was washed with the same buffer until A280 is stable and close to the base line. The proteins bound to the CM-Sepharose were eluted with a linear gradient of increasing sodium chloride (0 to 0.6 M). Fractions containing the proteins of interest were pooled and the A280 adjusted to 0.3 for DHBV Core Chimeric antigen vaccine and 0.2 for DHBV Core. Samples were dialyzed against 1 L of 20 mM sodium phosphate, 300 mM sodium chloride, pH 7.4, with three changes of the buffer.

3. Immunological Characterization of DHBV Chimeric antigen vaccines

The following sections describe the methodology used for the isolation of duck PBMCs and the evaluation of the binding of the DHBV Chimeric antigen vaccines to duck PBMCs.

3.1 Isolation and Culturing of Duck PBMCs

Duck PBMCs were isolated from whole blood by Ficoll density gradient separation. Cells were then cultured overnight in 100 mm culture plates in Iscove' s Modified Dulbecco' s Media (I-DMEM) with 10% FBS, at 37° C. Non-adherent cells were removed by washing the plates extensively with I-DMEM/10% FBS. Adherent cells were mechanically removed from the plate, washed in Dulbecco's PBS with 0.1% BSA and used for the evaluation of the binding of Chimeric antigen vaccines.

3.2 Binding of Chimeric Antigen Vaccines to Duck PBMCs

Adherent PBMCs were plated into the wells of a 96-well plate at 2×10⁵ cells/well. Cells were then incubated in the presence or absence of DHBV Core Chimeric antigen vaccine, Core or TBD at 4° C. for 1 hr. In some cases, cells were pre-incubated with 3 mM purified murine IgG Fc fragments for 20 min at 4° C. before the addition of antigen. Following incubation with antigen, cells were washed, and incubated for 20 min with either anti-mouse IgG-biotin Ab or anti-DHBV-Core-biotin mAb. Subsequently, cells were incubated with Streptavidin-Cy-Chrome for 20 min, washed, and resuspended in DPBS with 2% paraformaldehyde. At least 20,000 events were acquired using a FACSCalibur (BD Biosciences) with analysis performed using CELLQuest software.

3.3 FACS Acquisition and Analysis

Data on positive cells was acquired using FACSCalibur (BD BioSciences) fitted with

Cellquest acquisition and analysis software (BD). A gate was made on the viable cell population as determined by the FSC and SSC scatter profile and ≥10,000 events were acquired. To determine the percentage of positive cells, the gate was set based on negative control treated cells (isotype control labeled or cells labeled with F(ab′)2 goat anti-mouse Alexa-488 alone).

The percent of specific positive cells was calculated as:

$$\frac{( \% positive cells test sample - \% positive cells control )}{100 - \% positive cells control} \times 100$$

The relative mean fluorescent intensity (MFI) was determined as: MFI of the test sample—MFI of the control sample.

3.4 Experimental Animals and Procedures

Post-hatch persistently DHBV-infected groups of ducks were used for the evaluation of the effects of DHBV Core Chimeric antigen vaccine. The following sections describe the selection of animals and the procedures used for these experiments.

3.4.1 Generation and Screening of Ducks Chronically Infected with DHBV

In order to produce persistent infection post-hatch, ducklings from an uninfected breeding colony were inoculated (i.v) with 100-200 μl of DHBV positive serum (>100 pg DHBV DNA/10 μl by dot blot). The birds were screened for DHBV infection by serum dot blot post-hatch day 1-3 and week 3 and the animals with positive viremia were chosen for the study. Animals which were DHBV DNA dot blot positive on initial screen, those with DHBV viremia <10 pg/10 μl on week 3 and those with other obvious health problems were excluded from the study.

3.4.2 Determination of Serum DHBV DNA by DNA Dot Blot

10 μL of serum was applied to a Hybond-N+nylon membrane (Amersham Biosciences UK Limited) with alkali denaturation and ultraviolet cross-linking. Membranes were pre-hybridized for 2 hours and hybridized for 16 hours in 7%SDS/6×SSC at 65° C. DHBV probe was made using the Random Primer Kit (Thermo Fisher Scientific) from a monomer copy of the DHBV DNA in the plasmid pAltD2-8. Membranes were washed 2 times for 15 min in 0.1% SDS/2× SSC, followed by 215 min washes in 0.1% SDS/0.2× SSC. Signals were quantified based on comparison with plasmid standards of known concentration applied to the same membrane.

3.4.3 Antibody Levels in Duck Sera

The levels of antibodies in the duck sera were estimated by ELISA methods. ELISA plates were coated with either recombinant DHBV core antigen or DHBV PreS antigen (1 μg/mL, 100 μl/per well), overnight at 4° C. Plates were washed with washing buffer, followed by blocking with 2% BSA/PBS at RT for one hour. All samples were diluted with 2% BSA/PBS (1:50), 100 μl in each well, incubated at 37° C. for one hour, washed and incubated with Goat anti-duck IgG-HRP (1:4000) at 37° C. for one hour. The plates were washed with the wash buffer and the chromogen ABTS (KPL, MD, USA: 2,2′-azino-di-(3-ethylbenzthiazoline-6-sulfonate) was added into each well (100 μl/well) and the plates were incubated at RT for 10 min. The intensity of the developed color was measured at 405 nm.

4 Vaccination Protocol

4.1 The Effect of DHBV Core-TBD (Chimeric antigen vaccine) in Ducks with Established Persistent DHBV Infection.

Post-hatch DHBV-infected groups of ducks were used for these experiments. The experimental group received DHBV Core-TBD Chimeric antigen vaccine (20 μg in 150 αl buffer) as subcutaneous injections once every week for the first five weeks and 40 μg in 300 μl buffer once every four weeks until the termination of the experiment. The control group received injections of equal volume of the buffer (20 mM NaH₂PO⁴, 300 mM NaCl, pH 7.4) according to the same regimen as the other groups. The ducks were at the age of 3-4 weeks at the start of the vaccinations. The termination was at 33 weeks.

4.2 The Effect of Treatment with Lamivudine and Vaccination with DHBV Core Chimeric Antigen Vaccines in DHBV-infected Ducks.

DHBV-infected ducks (3-4 weeks of age) were treated with 3TC (lamivudine) by intramuscular injections (20 mg/kg/day, twice per day) for 16 days before initiation of vaccinations and continued for 10 weeks at this concentration. Starting at week 12 of the study, lamivudine dose was increased to 40 mg/kg/day in the same volume of buffer and continued for 4 weeks to achieve complete viral suppression as monitored by DHBV DNA dot blot.

The first group received subcutaneous injections of DHBV Core Chimeric antigen vaccine (40 μg in 300 μl buffer) once every two weeks, the first injection given two weeks after the initiation of lamivudine treatment. The second group received subcutaneous injections of DHBV Core (20 μg in 300 μl buffer) once every two weeks, the first injection given two weeks after the initiation of lamivudine treatment. The third group received injections of the buffer (20 mM NaH₂PO₄ pH 7.4, 300 mM NaCl). The total duration of the experiment was 24 weeks.

4.3 Collection of Tissue Samples

Blood (0.1-0.2 ml) was collected from post-hatch DHBV-infected ducks before the infection, at weeks 1 and 3 following the infection for the evaluation of DHBV viremia and once every two weeks (1 ml). On completion of the study, liver and spleen samples were snap frozen in liquid nitrogen. For the evaluation of histology and toxicology, the following tissues were preserved in formaldehyde: liver, spleen, lungs, kidneys, heart, pancreas, duodenum, injection and non-injection site skin and muscle.

4.4 Outcome Measures

The outcome of the study was evaluated by measuring the serum DHBV DNA levels by dot blot (section 3.4.2) and the antibody responses (Section 3.4.3). Serum SGPT (ALT) and other enzymes in the duck sera were estimated using automated assays carried out under contract with CVPLD Veterinary Pathology Laboratory in Edmonton. Histology, pathology and H+E staining of all tissues an immunohistochemistry assays were done by a contract research organization (Histobest, Edmonton), using antibodies against DHBV core and PreS proteins.

5. RESULTS

5.1 Antigen Binding Receptors on Duck PBMC

In mammalian systems there are several routes for entry of an antigen into an antigen presenting cell, especially DCs. Among these, the receptor-mediated uptake is of prime importance. CD64, also known as FcγRI, is a high affinity receptor which functions as an efficient uptake mechanism for IgG complexed antigens. Following uptake, the antigen can be processed and presented through class I and class II pathways. CD32, also known as FcγRII, is a low affinity receptor for the efficient uptake of IgG-complexed antigens. The antigen can be processed and presented through class I and class II pathways. CD16, also known as FcγRIII, is a low affinity receptor for IgG of which the details of antigen processing and presentation are not well characterized. CD206 is a high affinity receptor for antigens with high mannose content and the processing is mainly through the class II pathway. In addition to these receptor-mediated mechanisms of uptake of antigens, the other major pathway for antigen uptake is by phagocytosis and macropinocytosis. The mechanism of antigen uptake in avian species and especially in ducks is not well characterized (Schultz et al., 2004). It has been suggested that there are some similarities between the general mammalian and avian immune systems with some very important differences (Sharma, 1997). Crystal structures of Fcγ receptors have identified the binding regions of Fc fragments from different species and duck receptors seem to have very similar recognition sites (Sondermann et al. 2001). Based on this observation we have carried out some studies evaluating the binding of DHBV chimeric antigen vaccine to duck PBMCs.

5.2 Binding of DHBV Chimeric Antigen Vaccines to Duck PBMCs: Analysis by Flow Cytometry

We have investigated the ability of DHBV Chimeric antigen vaccines to bind to adherent duck PBMCs. PBMCs were isolated from duck blood by Ficoll density gradient separation and cultured for 20 hr in I-DMEM/10% FBS at 37° C.

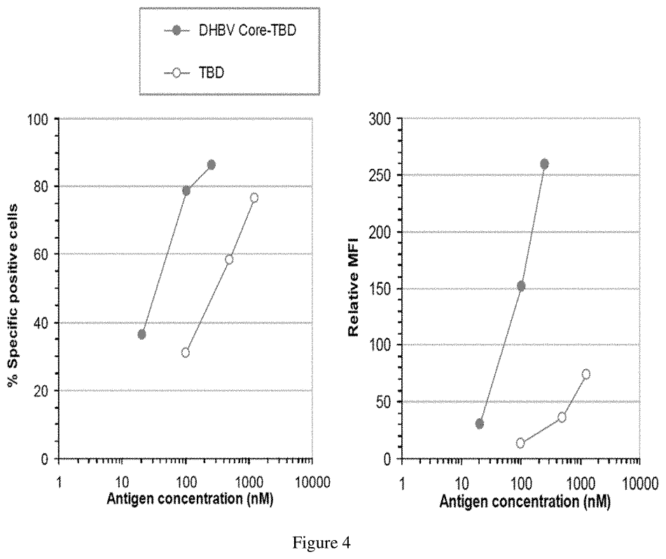

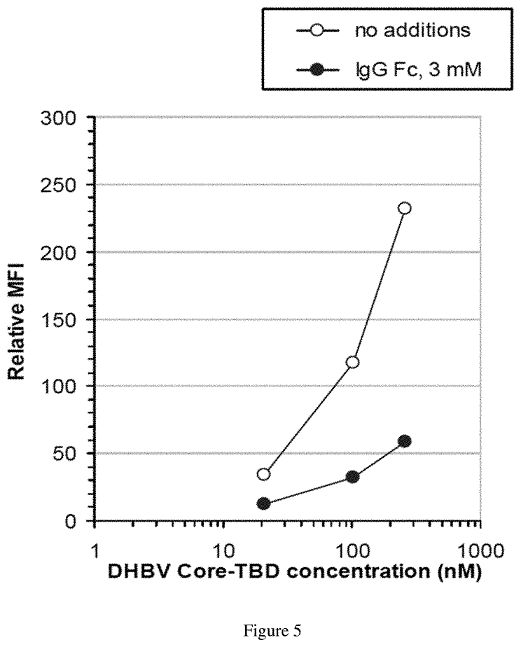

The culture plate was washed to remove non-adherent cells and the adherent cells were removed by mechanical detachment and used for binding studies. The cells were incubated with the chimeric antigen vaccine (antigen) in PBS/0.1% BSA for 1 hr at 4° C. The binding of the TBD domain was recognized with goat anti-mouse IgG biotin and SA-Cy, the core domain recognized with anti-duck core-biotin mAb, followed by goat anti-mouse Fab IgG FITC Ab. Data acquisition was by flow cytometry in order to determine the % positive binding cells and relative MFI. The results are presented in FIG. 4 show that the fusion protein is able to bind to the duck PBMCs. This binding was inhibited by Fc fragments, suggesting the involvement of Fc-specific receptors on the duck cells for the binding of the Chimeric antigen vaccine (FIG. 5).

5.3 Evaluation of the Effect of Chimeric Antigen Vaccines in Chronically DHBV-infected Ducks

Experiments were carried out in post-hatch DHBV-infected (persistent) ducks. DHBV Core Chimeric antigen vaccine was tested in post-hatch infected ducks (persistent infection) in the presence and absence of pretreatment with lamivudine. The following sections describe the results from these studies.

5.4 Evaluation of the Effect of DHBV Core-Chimeric Antigen Vaccine in Persistently DHBV-infected Ducks

Ducks persistently infected with DHBV were treated with DHBV Core-TBD Chimeric antigen vaccine or equivalent amount DHBV Core protein, as described previously and the control group received the buffer used for the injections.

5.4.1. Significant Decrease in Viremia was Observed in Ducks Treated with Core Chimeric Antigen Vaccine

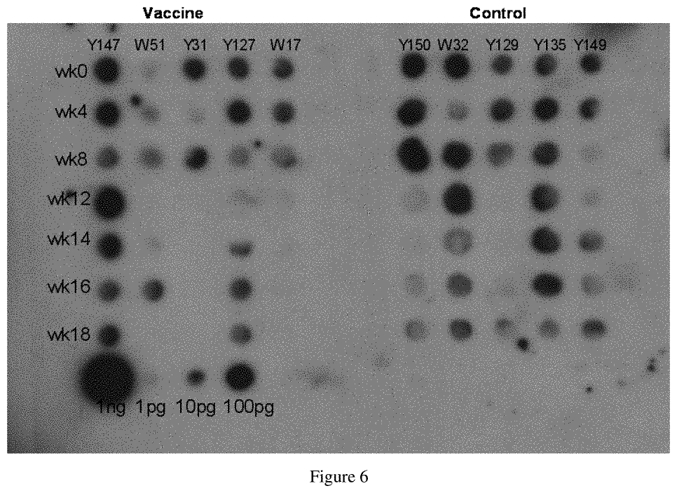

Blood samples were collected from persistently DHBV-infected ducks at different time points during the treatment and the DHBV DNA levels were estimated by DNA dot blots. In some of the post-hatch DHBV-infected ducks, a decrease in serum DHBV DNA was observed up to week 18 of the treatment with Core-Chimeric antigen vaccine, but this effect was not very evident at later time points (FIG. 6). In addition, in the control group there was a decrease of viremia in some animals. If these effects are due to spontaneous clearance of the virus is unclear.

5.4.2 Anti-Core Antibody Levels Showed an Increase Following Vaccination in persistently DHBV-infected Ducks

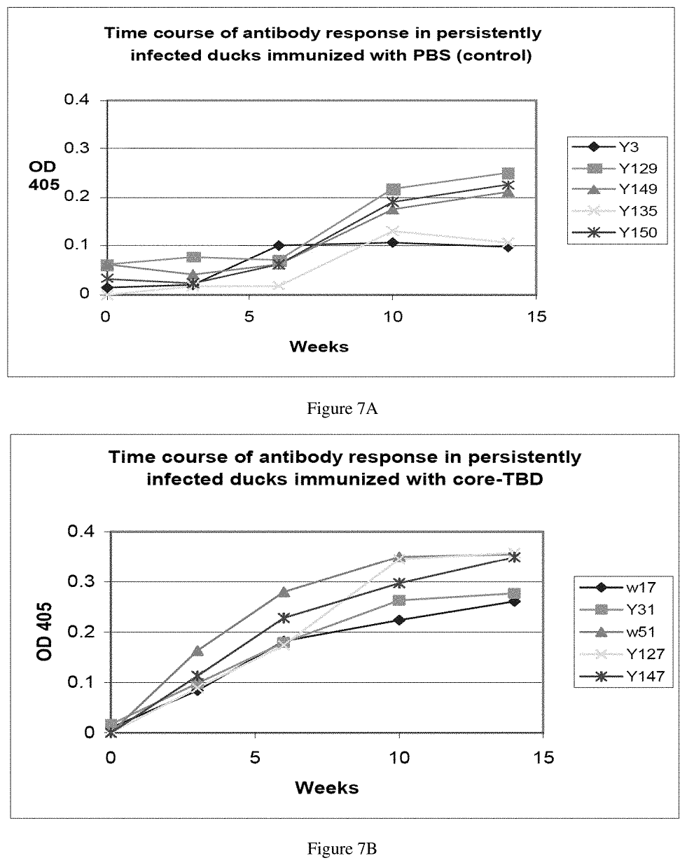

Anti-core antibody levels were measured in the serum from ducks collected at different time points during the therapy as described previously. The results from the persistently infected group in FIG. 7B show that there was an increase in the antibody levels in comparison with the control group in FIG. 7A.

6. Combination therapy: Evaluation of the Effect of Core-Chimeric Antigen Baccine in Chronically DHBV-infected Ducks Pre-treated with Lamivudine (3TC)

It has been suggested that one of the mechanisms by which the HBV induces tolerance in the host is by the production of large quantities of virus and viral proteins to saturate the host immune machinery. By inference, a decrease in viremia and the resulting viral antigen concentration would be beneficial in generating immune responses during therapeutic vaccination.

This possibility was tested in post-hatch DHBV infected ducks pretreated with 3TC, as described above. The results from these experiments are presented in the following sections.

6.1 The Effect of 3TC Treatment Followed by the Administration of DHBV Core-Chimeric Antigen Vaccine in Persistently DHBV-infected Ducks

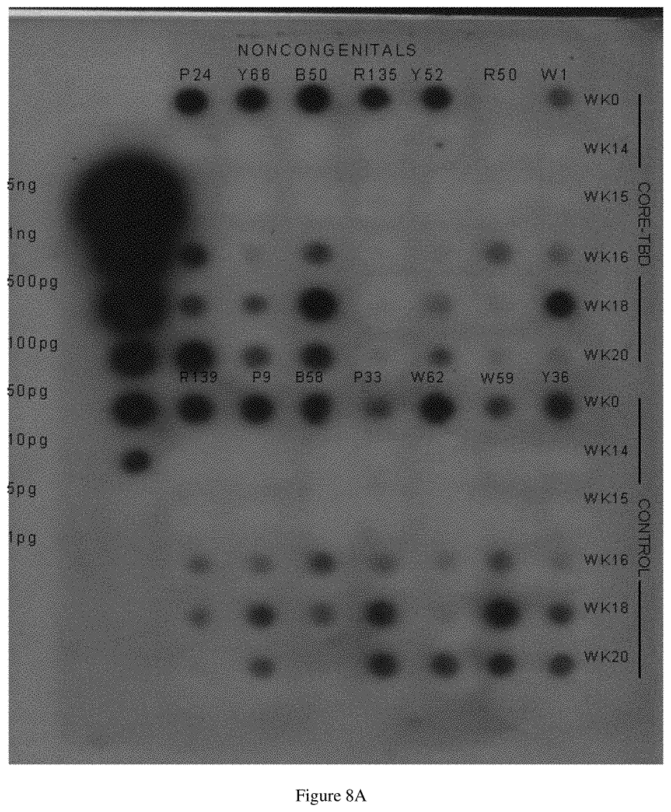

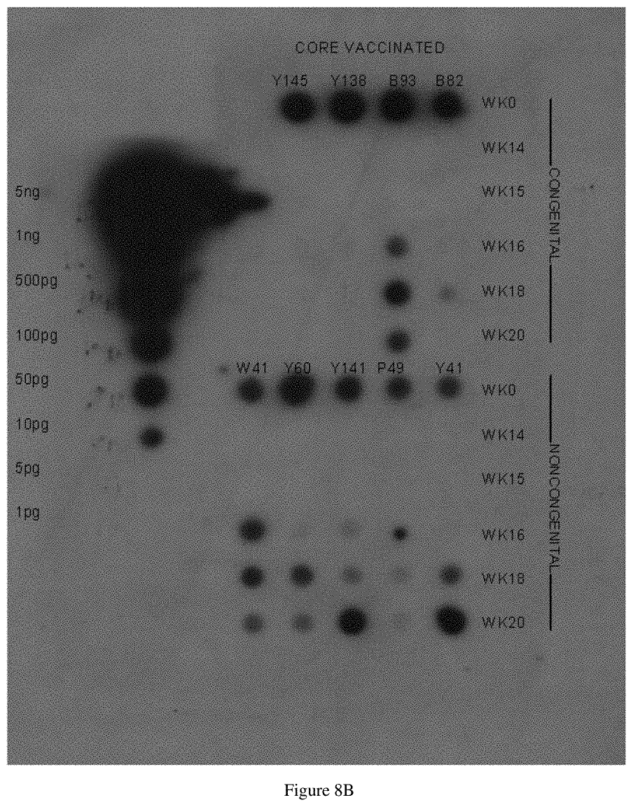

Ducks were pre-treated with 3TC, followed by immunizations with DHBV Core chimeric antigen vaccine, as described previously. DHBV viremia in the duck sera, as determined by DHBV DNA dot blots, is presented in FIG. 8B in contrast with the control shown in FIG. 8A. The results show that there was a delay in the onset of rebound of viremia in some animals

6.2 Inflammatory Enzymes in the Liver of Vaccinated Ducks Showed No Significant Change as a Result of Vaccination

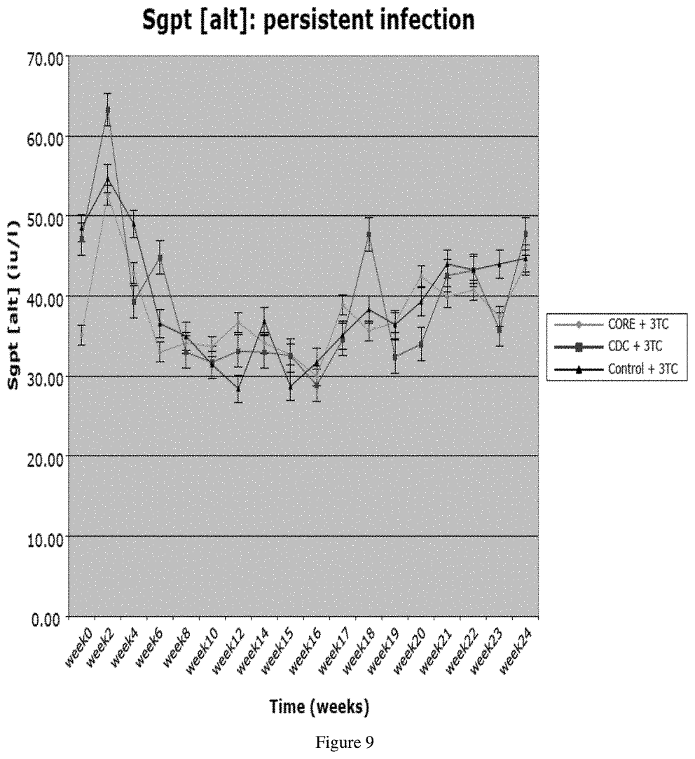

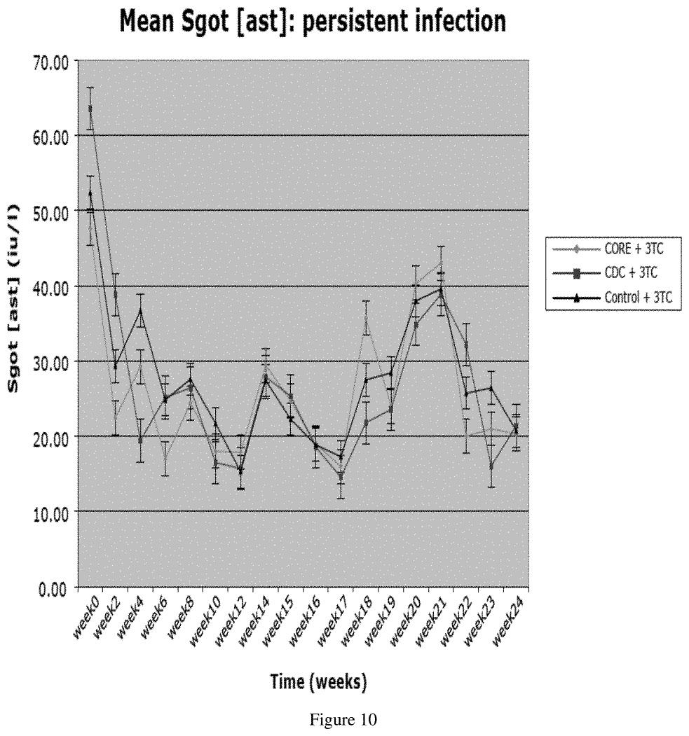

DHBV core-Chimeric antigen vaccine did not elicit a significant increase in the inflammatory enzymes in the post-hatch infected groups pre-treated with 3TC. Some of the results from these experiments are presented in FIGS. 9 & 10. The following liver enzymes were assayed in serum samples from the groups of ducks used in the study: SGOT (AST), SGPT (ALT), Sorbitol dehydrogenase-AO, Alkaline phosphatase and Gamma glutamyl transferase (GT). The results of ALT and AST estimations from three individual ducks from each of the treatment groups are shown in FIGS. 9 and 10 respectively. There was no noticeable difference in the treated groups compared to the controls, although there was significant fluctuation in the values in all the animals. There was no vaccine-related mortality among these groups of animals.

7. Summary of Results

In persistently DHBV-infected ducks, DHBV core chimeric antigen vaccine showed a decrease in viremia up to week 18 compared to controls, but this effect was not sustained at the longer time points. This probably is due to the lack of sustained (memory) T cells in the avian species. Combination therapy using 3TC and core chimeric antigen vaccine showed a delay in the onset of the rebound of viremia following 3TC withdrawal, compared to the control group. Persistently infected ducks showed an increase in antibody levels against DHBV core chimeric antigen vaccine. There was no noticeable adverse reaction to the vaccine in any animal and there was no morbidity or mortality associated with the administration of the chimeric antigen vaccine.

### EXAMPLE 2

### Evaluation of HBV Core Chimeric Antigen Vaccine as Immunotherapy for Chronic Infection (The Study was Carried out by NIH USA, Expt. No. NHA-77, October 2007)

1. Design of the HBV Core Chimeric Antigen Vaccine

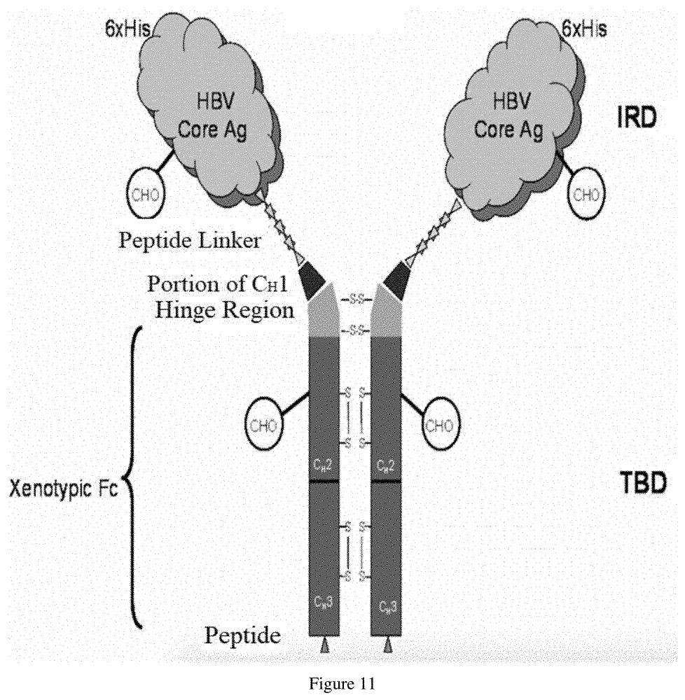

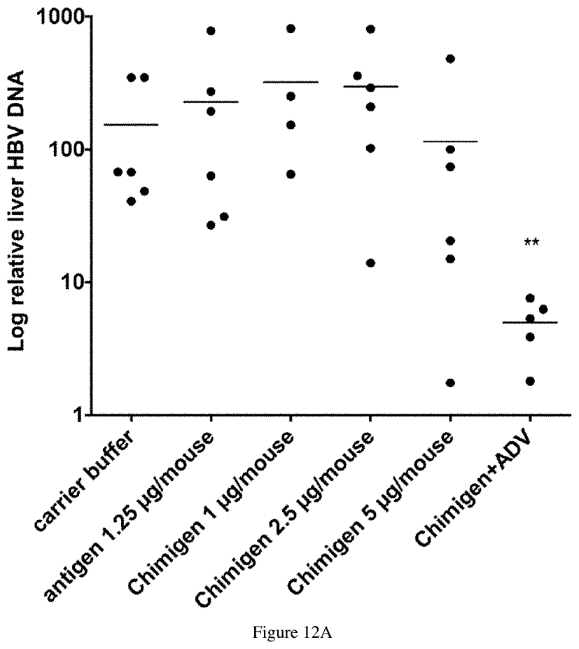

HBV Core chimeric antigen vaccine monomeric fusion protein can be schematically represented as: (N-terminus) 6xHis-rTEV Protease Site-HBV Core protein - - - [IRD] - - - Linker Peptide-Part CH1-CH2-CH3-Peptide (C-terminus) [TBD] as shown in FIG. 11.

2. Transgenic Mice Model for Chronic HBV Infection

HBV transgenic mouse is an experimental model developed to study HBV infection, virus replication and various treatment options, including immunotherapy. Immune responses to HBV Core chimeric antigen alone, and in combination with an HBV antiviral agent (Adefovir dipivoxil, ADV), was tested.

2.1 Materials and Methods

Animals:

Homozygous female and male transgenic HBV mice were used (22.2±2.8 g). The mice were originally obtained from Dr. Frank Chisari (Scripps Research Institute, LaJolla, Calif.) (Guidotti et al., 1995) and were subsequently raised in the Biosafety Level 3 (BL-3) area of the AAALAC-accredited USU Laboratory Animal Research Center (LARC). The animals were derived from founder 1.3.32. Pre-experiment liver biopsies were obtained and assayed for liver HBV DNA, and subsequently block-randomized into treatment groups. This study was conducted in accordance with the approval of the Institutional Animal Care and Use Committee of Utah State University with an expiration date of 8 Jun. 2008. The work was done in the AAALAC-accredited Laboratory Animal Research Center of Utah State University. The U. S. Government (National Institutes of Health) approval was renewed 27 Feb. 2002 (Assurance no. A3801-01) in accordance with the National Institutes of Health Guide for the Care and Use of Laboratory Animals (Revision; 1996).

Mice not allowed to wean before 3 weeks of age, i.e., not allow to eat solid food, have consistently higher liver HBV DNA titers. Use of such mice avoids the entry of animals that express liver HBV DNA at the limits of detection. Consequently, we have entered mice that were not weaned early and that were biopsied to obtain a pre-experiment liver HBV DNA value.

Compounds:

The HBV Core chimeric antigen vaccine was prepared following directions supplied above: the vaccine in liquid form was diluted with a carrier buffer (final dialysis buffer) to pre-described levels and stored at 2-8° C. for the length of the experiment.

Adefovir dipivoxil (ADV) was obtained from Gilead Sciences (Foster City, Calif.) by USU. Permission was obtained to use ADV as a positive control. The ADV was prepared in citric acid (0.05 M, pH 2.0), which was used to enhance the solubility of ADV in liquid suspension. It was administered by oral gavage in a 0.1 mL volume.

Liver HBV DNA assay:

Liver tissue was homogenized in lysis buffer immediately upon necropsy. The tissue (approximately 0.1 g) was ground with a well-fitted pestle in a microcentrifuge tube containing lysis buffer (1 mM EDTA, 10 mM Tris, 10 mM NaCl, 0.5% SDS, proteinase K). After incubation for 5-10 min at room temperature, the tubes were snap-frozen in liquid nitrogen for storage. For extraction of the DNA, the samples were incubated at 55° C. for 2 to 4 hours. Samples were poured into Phaselock™ Gel tubes containing 150 μL water, 350 to 500 μL phenol. After shaking and centrifuging at 12,000 rpm for 15 m, contents were poured into a second tube containing 350 to 500 μLt chloroform. After centrifuging, the DNA was precipitated with 50 μL 5 M NaCl and 650 μL isopropanol and washed with 70% ethanol. The dried pellets were suspended in 500 μL TE buffer containing 1 μL RNase and incubated overnight at 55° C. with occasional shaking. A specified volume of DNA solution, typically containing 40 μg, was digested with Hind III enzyme (New England Biolabs, Beverly, Mass.) for an incubation period of 3 h at 37° C. Hind III has been shown to not cut within the HBV transgene sequence. Digested DNA was separated by electrophoresis utilizing a 1% TAE-buffered agarose gel at 80 V for 3-4 h. DNA was then transferred to BioDyne™ B positive-charged nylon membrane by alkaline transfer method with the following modifications: 1) the gel was soaked in 0.4 M NaOH for 15-30 min, 2) the nylon membrane was soaked in water followed by 0.4 M NaOH for 5 min. The sponge used for transfer was also saturated in 0.4 M NaOH. The treated gel was placed (well-opening-side down) upon absorbent paper on the sponge. Transfer occurred over a 3 h period, after which the gel was discarded and the nylon membrane was washed in a solution of 0.2 M Tris (pH 7.6), 2× SSC, and 0.1% SDS. The membrane was baked for >30 min at 80° C. and UV—fixed using UV StratalinkerTM 1800 (Stratagene, La Jolla, Calif.). Prior to hybridization, the filter was rinsed twice for 30 min in a neutralizing solution of 0.1× SSC and 0.1% SDS. Hybridization using a [32P] CTP-labeled, HBV genomic probe (digested with Hae III) cloned into the pBluescript plasmid (gift of Dr. Luca Guidotti, The Scripps Institute, LaJolla, Calif.) occurred overnight at 60° C. in a solution of 10% PEG-8000, 0.05 M NaPO4, 0.21 mg/ml salmon sperm DNA, and 7% SDS.

The radioactive signals were measured using a Phosphor Imaging method (Optiquant). An image of the radioactive filter was exposed overnight to a Cyclone™ Storage Phosphor Screen (Perkin Elmer Multisensitive Medium, Type MS PPN 7001723). The exposed screen was transferred to the Cyclone™ drum and read using the 600 dpi setting. The ratio of the viral DNA bands to the transgene band was used to determine the concentration of viral DNA per host DNA. This calculation was based upon the knowledge that there are 1.3 copies of the transgene present per host cell with this line of transgenic mice (personal communication, F. Chisari). The transgene was used as an internal indicator to calculate the pg of HBV DNA per μg of homozygous cellular host DNA.

Liver HBcAg assay:

For detection of hepatitis B core antigen (HBcAg), liver biopsies were first paraffin-embedded. The paraffin was then removed from the sections by using two 5 min treatments with xylene. Tissues were fixed with two 3 min treatments with 95% ethanol. Sections were treated with deionized water for 3 min, exposed to 3% hydrogen peroxide for 5 min, and Biotin-block (#X0590, Dako Corporation) for 5 min. The primary antibody, rabbit anti-HBcAg (1:100 dilution) (#B0586, Dako Corporation), goat anti-rabbit secondary antibody (#k684 Dako, LSAB Peroxidase Kit), strepavidin peroxidase (#K684 Dako, LSAB Peroxidase Kit), and substrate-chromogen solution (3-amino-9-ethylcarbazole, AEC) were added for durations of 30, 30, 10 and 10 min, respectively. Sections were counterstained with Mayer's hematoxylin before being mounted.

Three different parameters were obtained from each tissue section. The first two measurements are based on the observation that cells surrounding the central veins of the liver are more strongly stained than those in other areas of the liver (personal observation), and that drug administered intraperitoneally should have ready access to the luminal cells of the veins. The first two parameters were obtained from counting cells surrounding central veins as follows. The total number of cells, the number of cells with stained nuclei, and the number of cells with stained cytoplasms were counted around central veins. The stained nuclei counts or the stained cytoplasm counts were divided by the total cells. Three central vein areas were counted with each slide sample. For the third parameter, a field, not in a central vein area, was counted for the total number of stained nuclei. One-quarter of the field was counted. Three such fields were counted per liver section. The identity of the samples was blinded to the person reading the slides.

Sera HBeAg:

Whole blood samples were obtained during necropsy by cardiac puncture. The whole blood was then transferred to heparin-containing vials and centrifuged for the collection of the serum component. Ten microliters of serum were then diluted into 90 μL of negative control serum, resulting in a 1:10 dilution of each sample. These samples along with a serial dilution of a positive control and a calibrator were run on an HBeAg-specific ELISA (International Immuno Diagnostics, Foster City Calif.) per manufacturer's instructions. Using the known PEI units for the calibrator, PEI units were formulated for the serial dilutions of the positive serum. A graph was generated and extrapolation was used to assign a PEI unit value with a high degree of confidence (R2 value of 0.9816) for each sample with a high degree of confidence (R2 value of 0.9816). The ELISA manufacture's cut-off was utilized.

Sera HBsAg:

The same serum samples collected and prepared for the HBeAg ELISA were also used for the HBsAg ELISA. Fifteen microliters of serum were diluted in the same manner and run on an HBsAg-specific ELISA (International Immuno Diagnostics, Foster City Calif.) per manufacturer's instructions. Interpretations were then made from both the manufacture's cut-off chart (with blank correction) and the manufacturer's cut-off formulae (without blank correction).

Cytokine array—plasma:

The same serum samples collected and prepared for the HBeAg and HBsAg ELISAs were also used for the serum cytokine assay. Five microliters of serum were diluted into 120 μL of sample dilution buffer, resulting in a 1:25 dilution of each sample. Thirty microliters of this prepared sample were then run on a Q-Plex™ Mouse Cytokine Array (Quansys Biosciences, Logan, Utah) per manufacturer's instructions. The fully developed cytokine plate was then captured as a .tif image on a Fuji LAS-3000 Luminescent Image Analyzer (Fuji Life Sciences, Stamford, Conn.T) and analyzed with Quansys Array Software™, version 1.3.

Cytokine array—liver and spleen:

Liver or spleen biopsies (approximately 35 mg each) were collected during necropsy and quickly homogenized in sterile PBS containing 0.1% NP-40. These homogenized samples were then snap-frozen in liquid nitrogen until the assay was performed. Just prior to performing the assay, the samples were rapidly thawed and centrifuged at 3000 rpm's for 20 minutes to remove any solid matter. The supernatant was then diluted 1:5 into sample dilution buffer and run on a Q-Plex™ Mouse Cytokine Array (Quansys Biosciences, Logan, Utah) per manufacturer's instructions. The fully developed cytokine plate was then captured as a .tif image on a Fuji LAS-3000 Luminescent Image Analyzer (Fuji Life Sciences, Stamford, Conn.) and analyzed with Quansys Array Software™, version 1.3.

Plasma interferon-gamma assay:

An IFN-gamma ELISA kit (BD OptEIA ELISA Set—mouse IFN-g kit, cat.# 551866) was used to assay plasma collected on days 1, 14, 35, and 56 of the first vaccination.

Plasma chemistry panel:

A VetScan® Chemistry, Electrolyte and T4 Analyzer, specifically designed for veterinary medicine, was used in our BL-2 laboratory to process samples for the “comprehensive diagnostic profile,” which consists of ALT, BUN, creatinine, total bilirubin, albumin, alkaline phosphatase, globulin, glucose, Na+, K+, phosphorous, and total protein. Protocols with the instrument were used.

Liver HBV RNA:

Real-time RT-PCR was used to assay HBV-specific RNA in liver biopsies. Total RNA from tissues was extracted using Trizol™ reagent. Primer-pairs (HBV3 forward ATAAAACGCCGCAGACACATC (SEQ ID NO: 5), HBV3 reverse AACCTCCAATCACTCACCAACC (SEQ ID NO: 6) and HBV3 Taq-man probe [6˜FAM]-AGCGATAACCAGGACAAGTTGGAGGACA(SEQ ID NO: 7)-[BHQ1a-6FAM] were used. A second primer-pair (HBV4 forward GGACAAACGGGCAACATACCT (SEQ ID NO: 8), HBV4 reverse TCTTCCTCTTCATCCTGCCTGCT (SEQ ID NO: 9) and HBV4 Taqman probe [6˜FAM]TCCAGAAAGAACCAACAAGAAGATGAGGCA (SEQ ID NO: 10) [BHQ1a-6FAM] was used, without a noticeable difference between the two sets, so the HBV3 probe/primer set was used. A duplex reaction was done with the internal control, mouse GAPDH primers/probe. The primers and probe were the forward GCATCTTGGGCTACACTGAGG (SEQ ID NO: 11), reverse GAAGGTGGAAGAGTGGGAGTTG (SEQ ID NO: 12), and probe [5˜HEX]-ACCAGGTTGTCTCCTGCGACTTCAACAG(SEQ ID NO: 13)-[BHQ1a-5HEX]. The one-step FullVelocity™ QRT-PCR Master Mix (Stratagene, La Jolla, Calif.) was used for RT and amplification of HBV RNA and mouse GAPDH, with primers and probe at a final concentration of 0.1 μM. Two microliter of total cellular RNA extracted from infected or control tissues was used. Samples were run on a DNA Engine Opticon 2 (MJ Research Inc, Waltham, Mass.). A 25 μL reaction consists of 12.5 μL FullVelocity™ QPCR Master Mix, 0.375 ul dilute reference dye (1:500), 0.25 ul Stratascript™ RT/RNase Block Enzyme Mixture, and 0.5 ul FullVelocity™ Enzyme. The reaction contained 0.25 μL of both HBV-primers, 0.25 μL of both GAPDH-primers, and 0.25 μL of both probes, all having a stock concentration of 10 μM. Reverse transcription of cellular RNA were performed for 30 min at 50° C. followed by PCR, which consisted of 1 cycle of 2 minutes at 95° C., then 40 cycles of 10 sec at 95° C. and 30 sec at 60° C. The assay was run with a series of 10-fold dilutions of pooled liver RNA from HBV transgenic mice to obtain a standard curve. The y axis was the log dilutions of the standard, and the x axis was the C(t) values. R2 values were used to measure the quality of the curve, which was always above 0.098. Mean C(t) values were obtained for duplicates of each sample. The mean C(t) values of each sample were used to obtain the log relative RNA value using a formula of the fit line of the standard curve.

Statistical analysis:

One-way analysis of variance was done with Neuman-Keuls Multiple Comparison test using Prism 4 (GraphPad Software, Inc.).

2.2 Experimental design

The study design is identified in Table 1. HBV transgenic mice having measurable HBV DNA expression in pre-experiment liver biopsies were randomized between groups. At 0, 21, and 42 days, animals were treated intradermally with one of seven treatment groups indicated below. Heparinized plasma was collected on days 14, 35, and 56, which is two weeks after each vaccination. On day 56, mice were euthanized before collection of liver and the final plasma. Plasma was assayed for IFN-gamma (shipped to supplier) and ALT (plasma HBV antigens if enough volume left). Liver was collected for HBV DNA and cytokine array. Three liver punches were snap-frozen as a backup.

TABLE 1
Experimental design

| #/Cage | Group # | Transgenic | Drug | Dosage | Treatment Schedule |
|---|---|---|---|---|---|
| 6 | 1 | Tg | carrier | — | Intradermal injection |
| | | | buffer | | days 0, 21, 42 |
| 6 | 2 | Tg | antigen only | 1.25 μg protein/mouse | Intradermal injection |
| | | | | | days 0, 21, 42 |
| 6 | 3 | Tg | HBV Core | 1 μg protein/mouse | Intradermal injection |
| | | | vaccine | | days 0, 21, 42 |
| 6 | 4 | Tg | HBV Core | 2.5 μg protein/mouse | Intradermal injection |
| | | | vaccine | | days 0, 21, 42 |
| 6 | 5 | Tg | HBV Core | 5 μg protein/mouse | Intradermal injection |
| | | | vaccine | | days 0, 21, 42 |
| 6 | 6 | Tg | HBV Core | 2.5 μg protein/mouse | Intradermal injection |
| | | | vaccine + | + 1 mg/kg ADV | days 0, 21, 42 |
| | | | ADV | | |
| 6 | 7 | Non-tg | HBV Core | | Intradermal injection |
| | | | vaccine | | days 0, 21, 42 |

2.3 Results

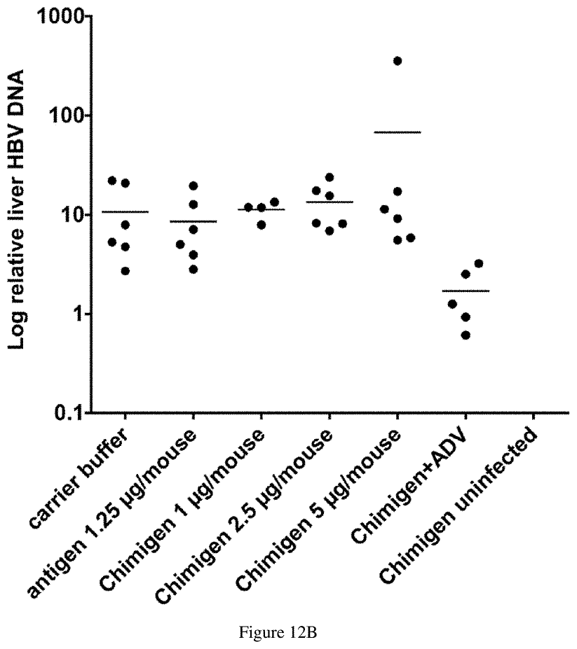

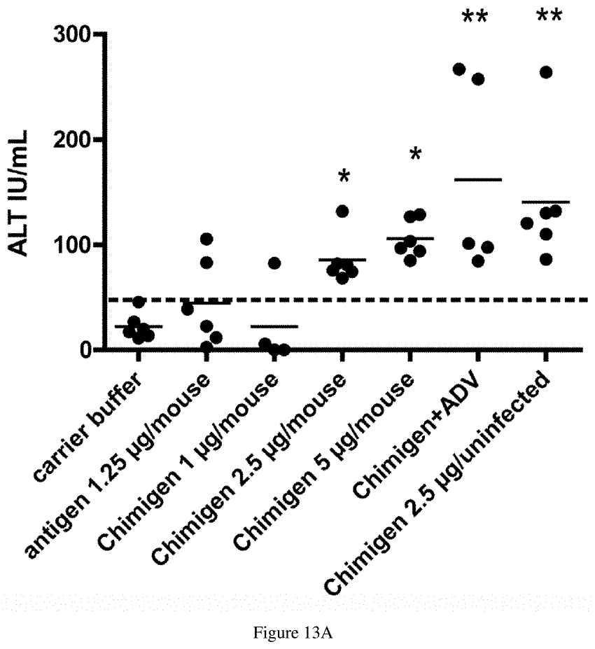

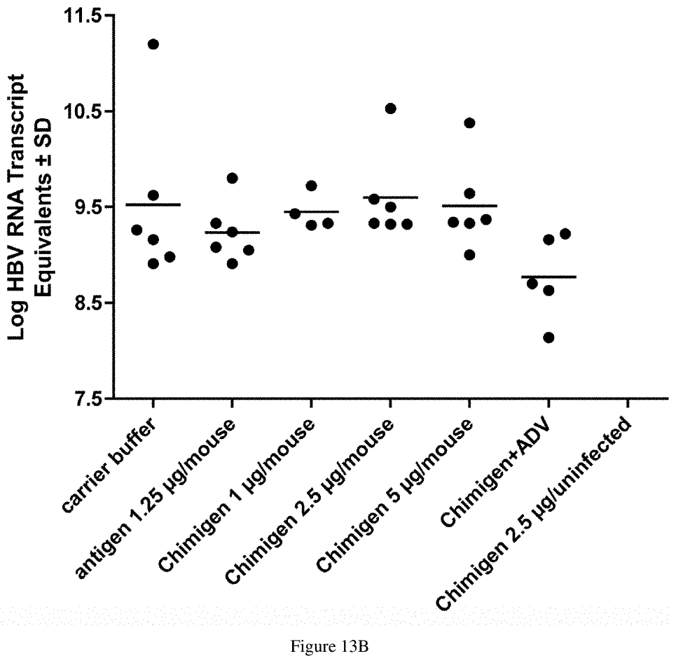

HBV Core chimeric antigen vaccine alone did not significantly affect liver HBV DNA, when measured either by real-time PCR (FIG. 12A) or by Southern blot hybridization (FIG. 12B). As expected, ADV at 5 mg/kg/day reduced liver HBV DNA. The HBV liver RNA was not significantly altered by vaccine (FIG. 13B). These results suggest that tolerance in transgenic mice was not broken with the vaccine.

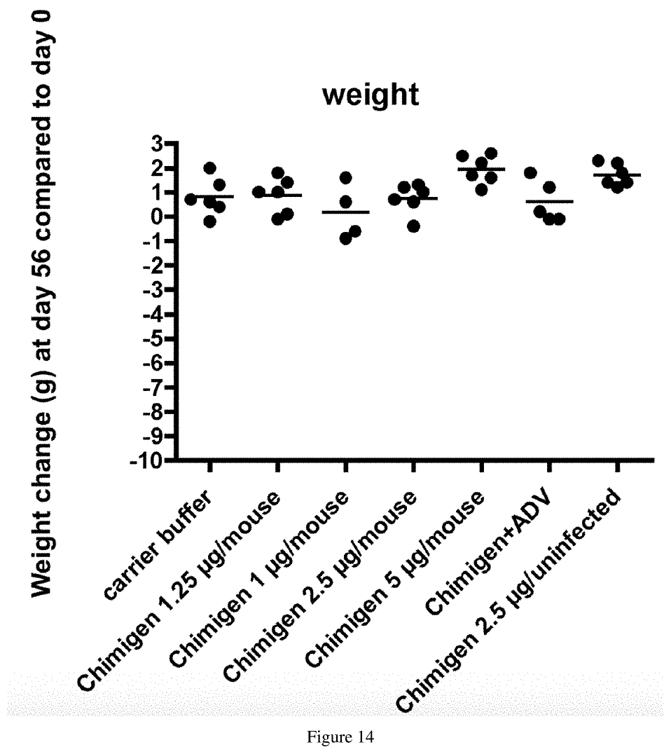

Plasma ALT was not increased in the animals treated with carrier buffer, but it was increased in animals treated with higher doses (2.5-5.0 μg/mouse) of HBV Core vaccine in both transgenic and non-transgenic mice, which suggests that the vaccine alone caused release of ALT from hematocytes (FIG. 13A). According to whole body weight change, however, the vaccine did not cause any toxicity (FIG. 14).

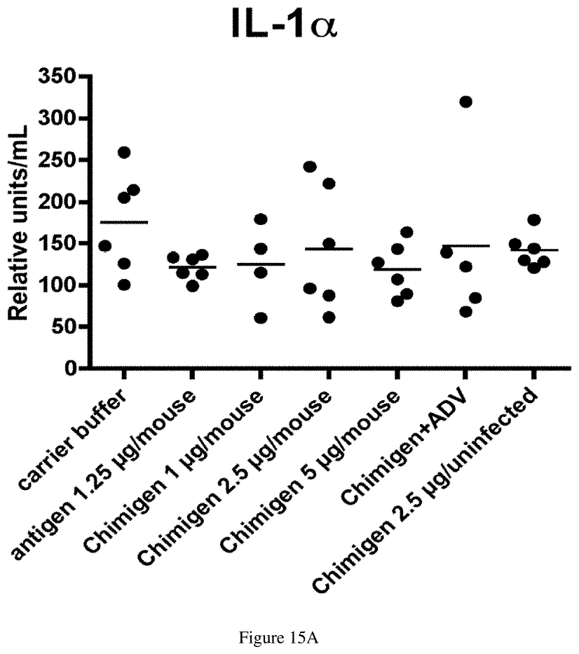

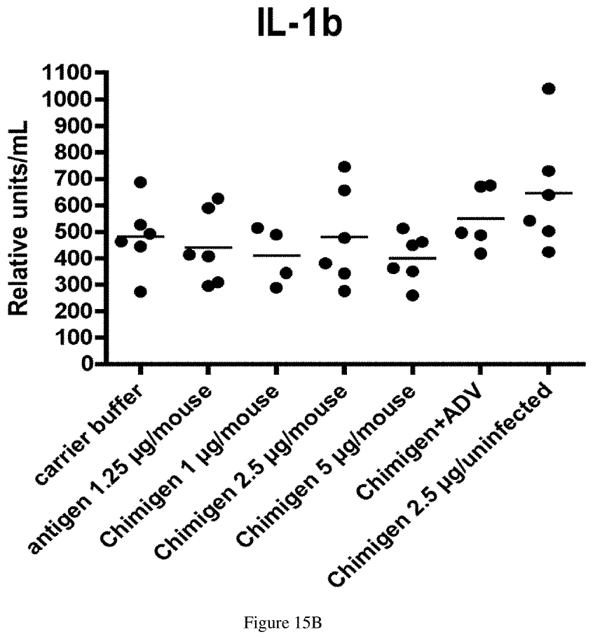

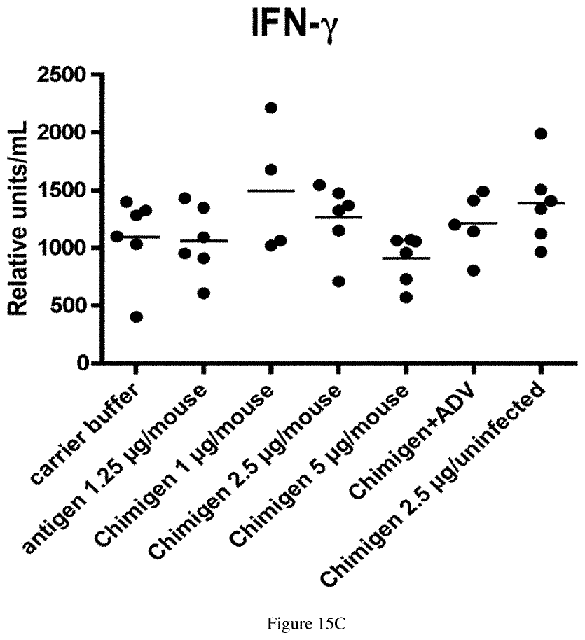

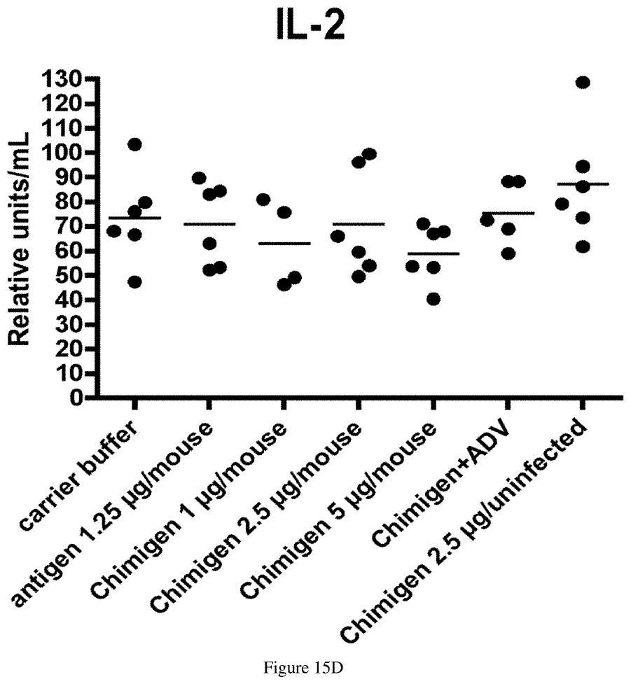

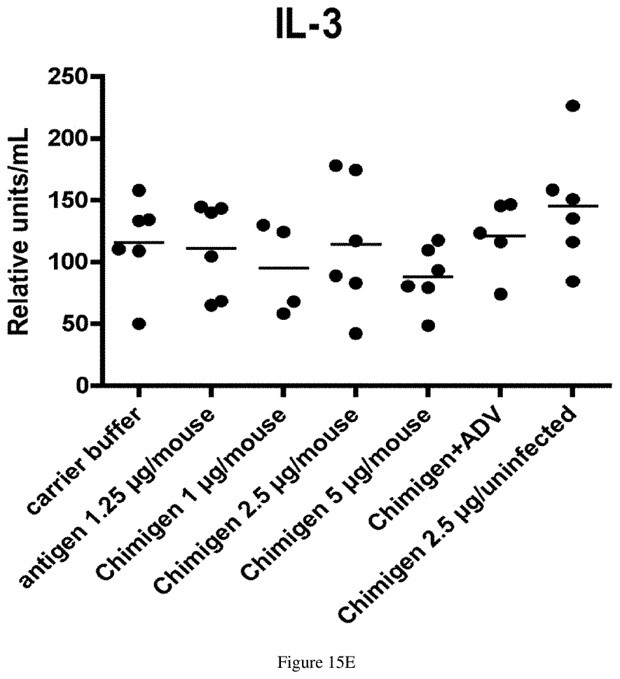

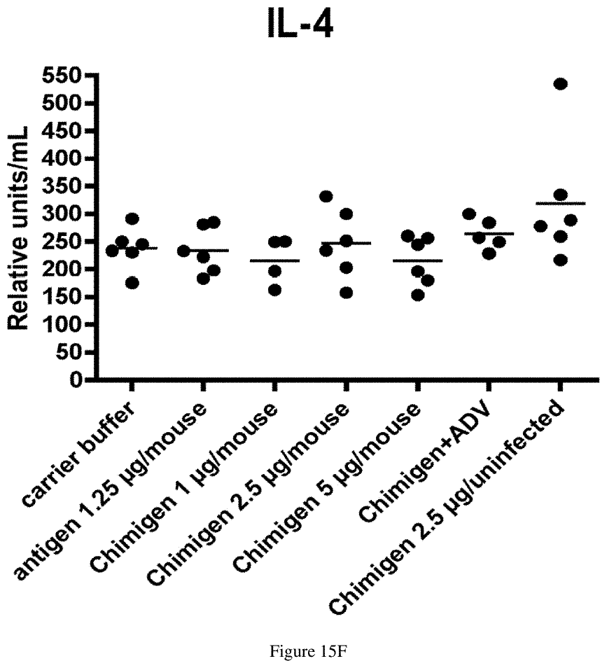

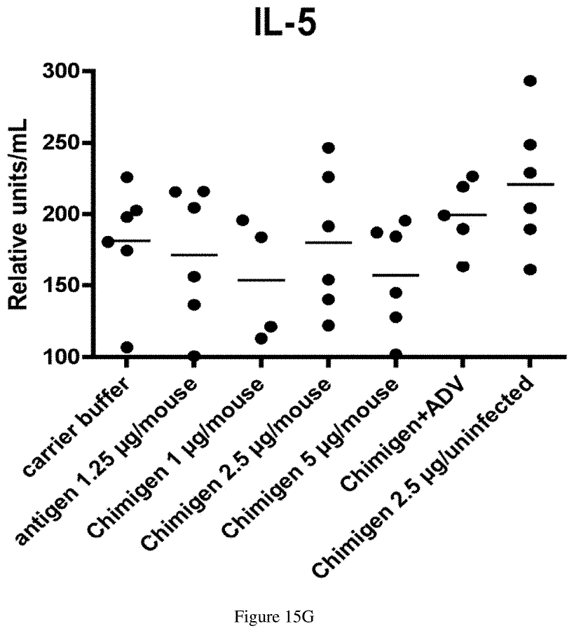

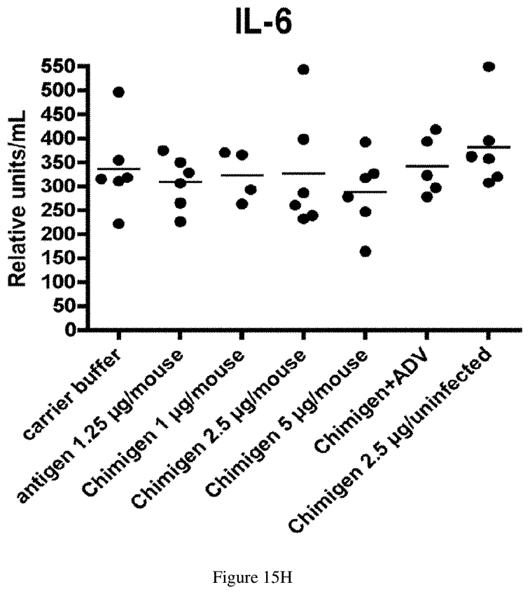

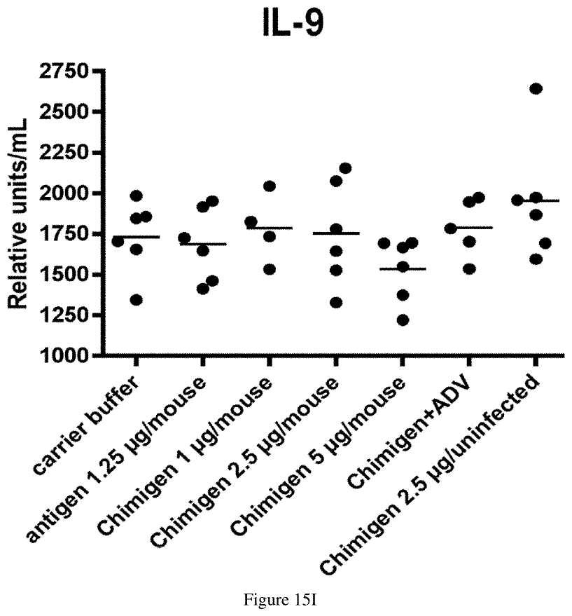

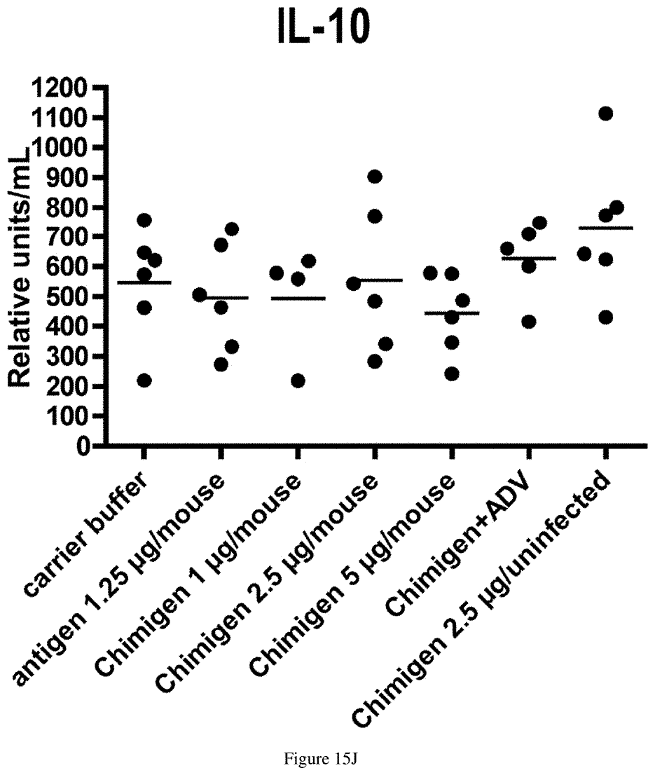

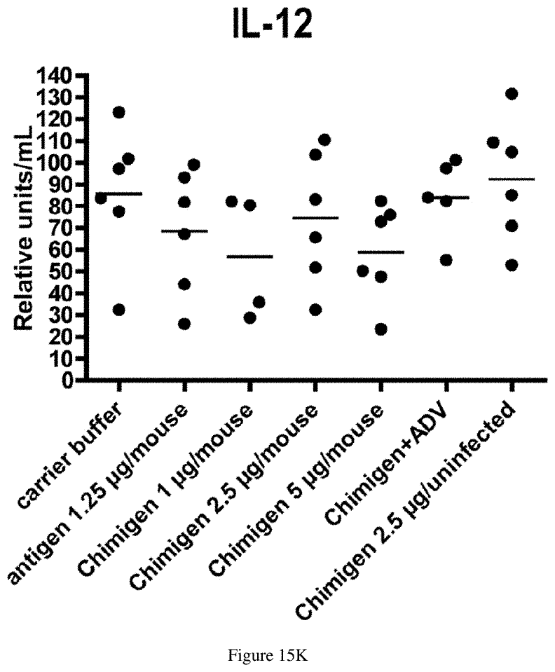

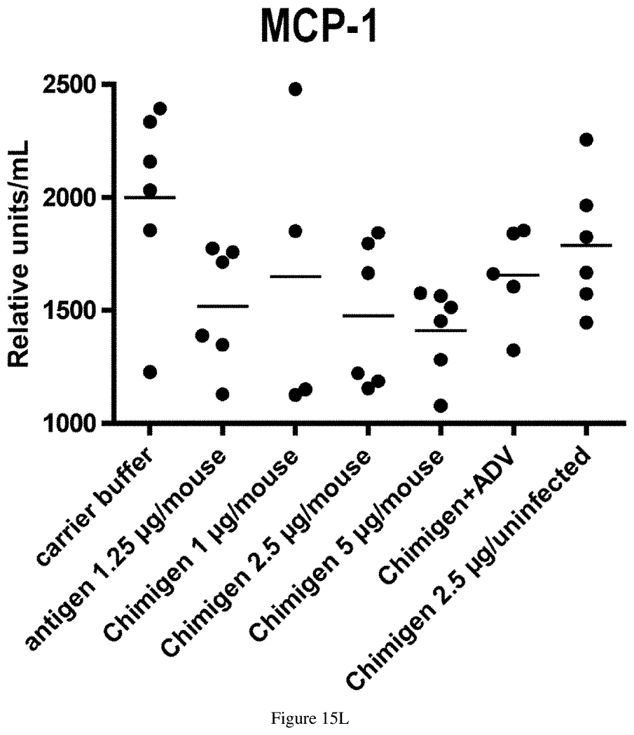

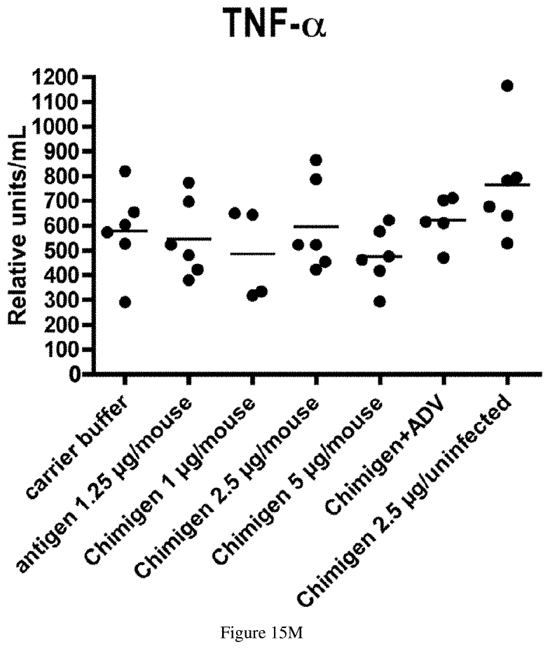

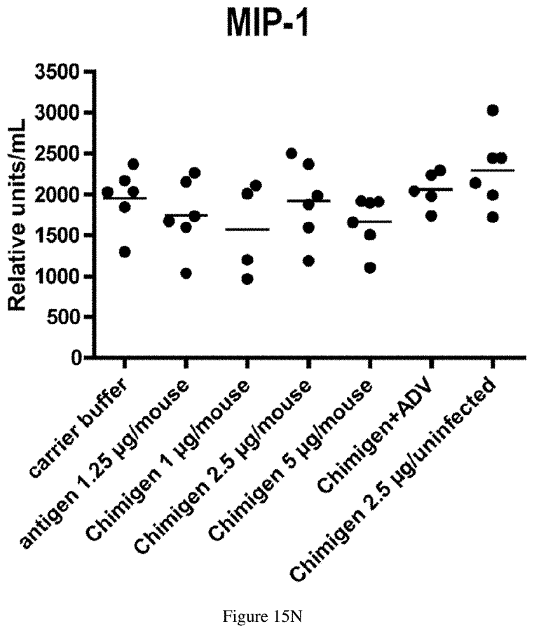

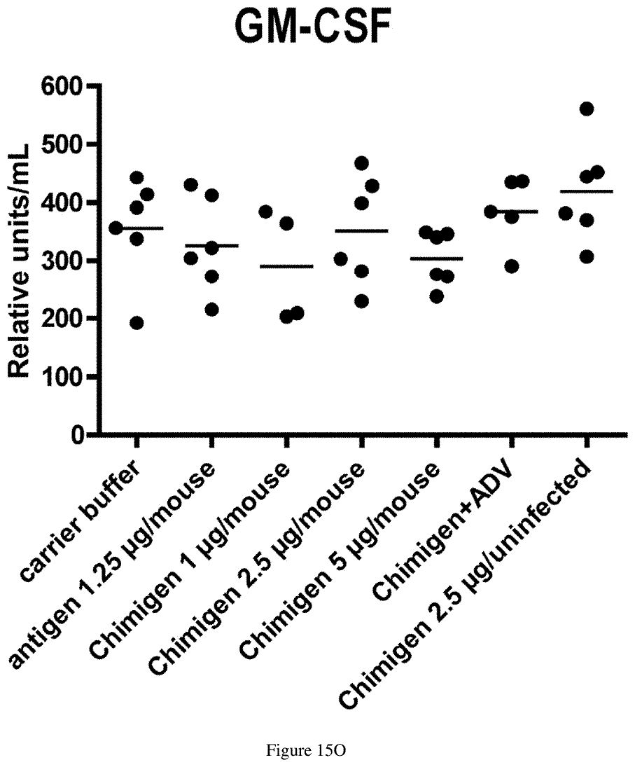

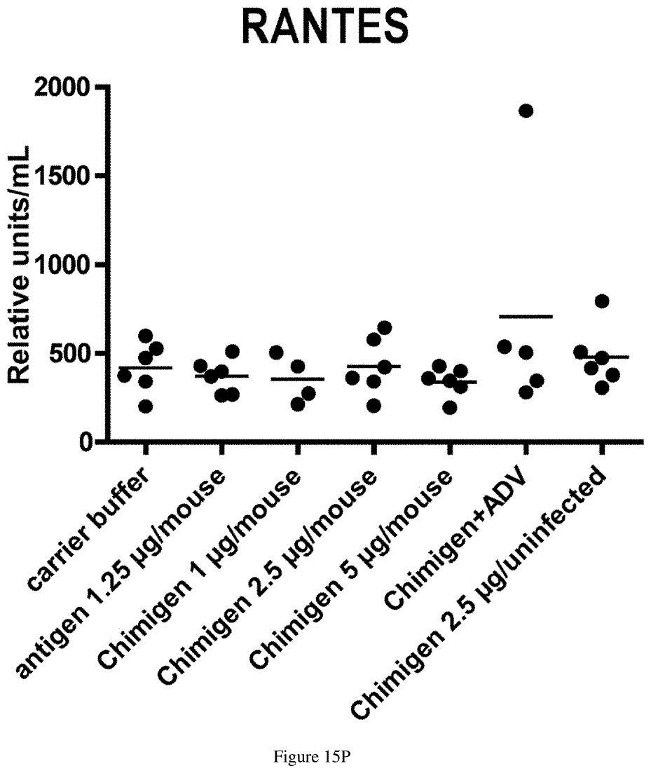

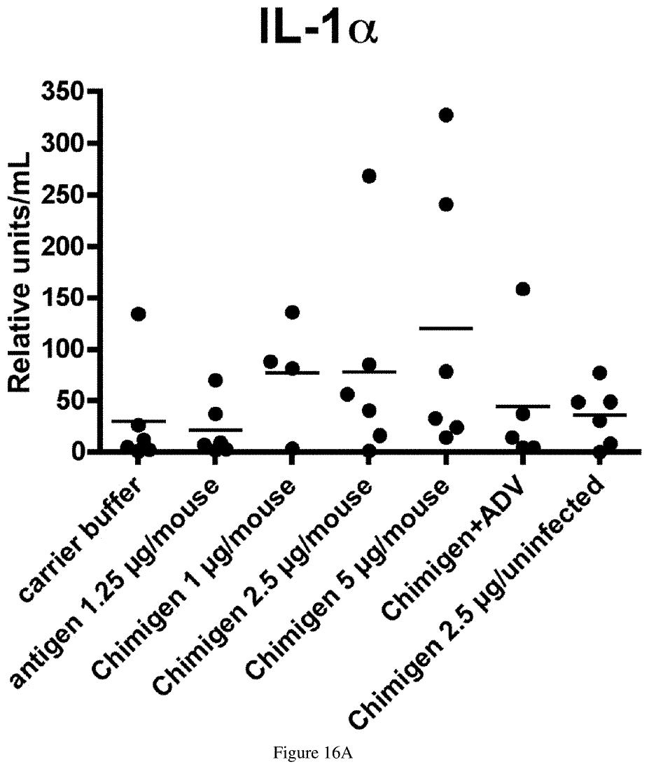

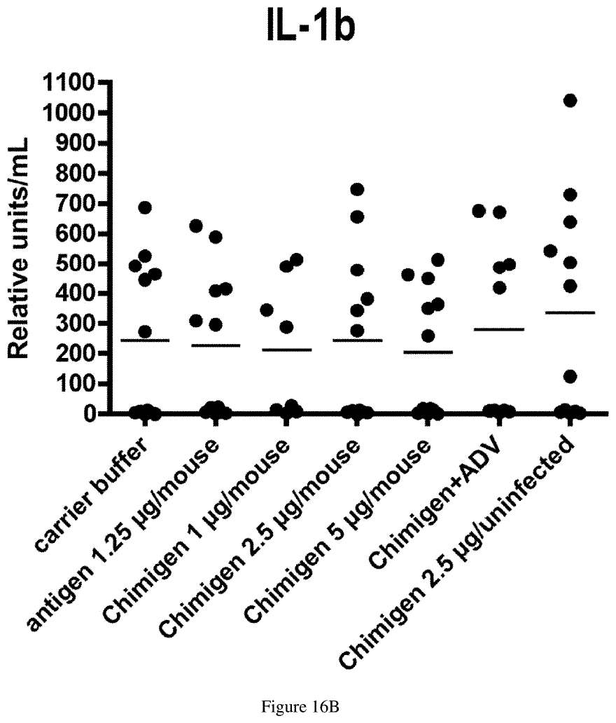

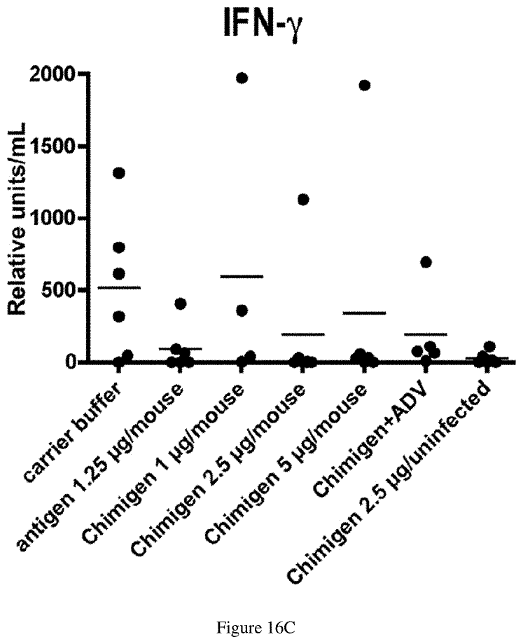

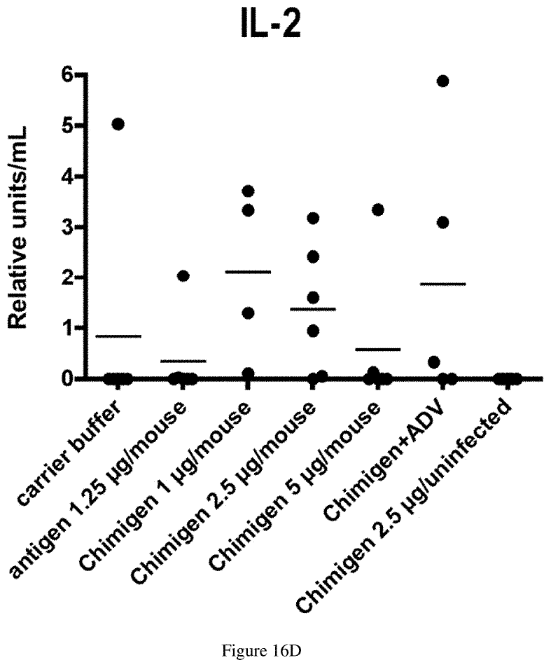

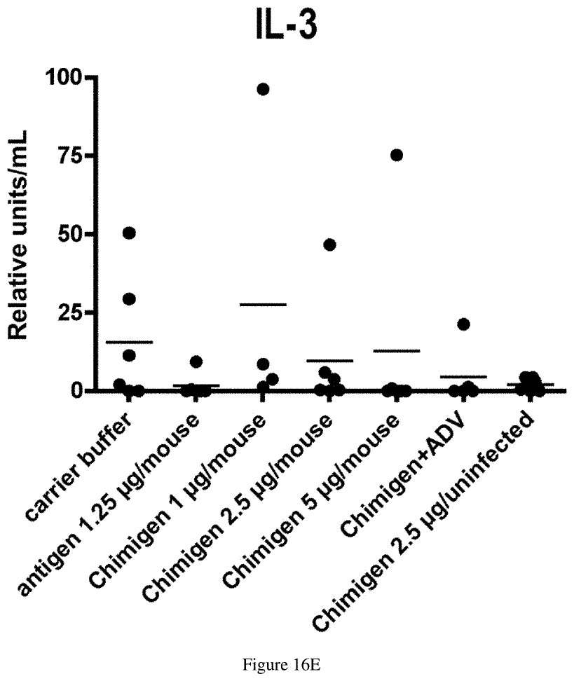

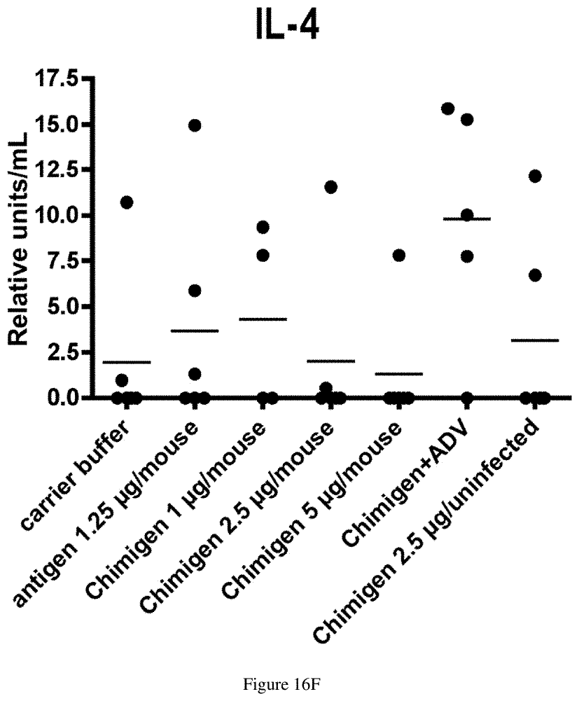

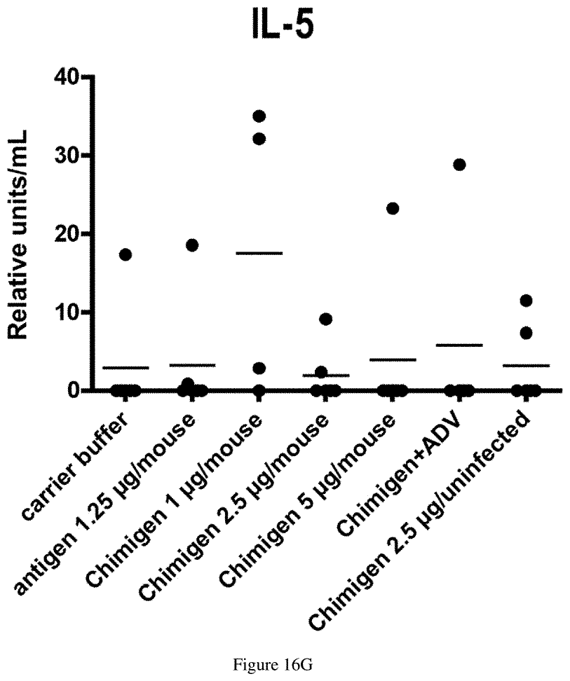

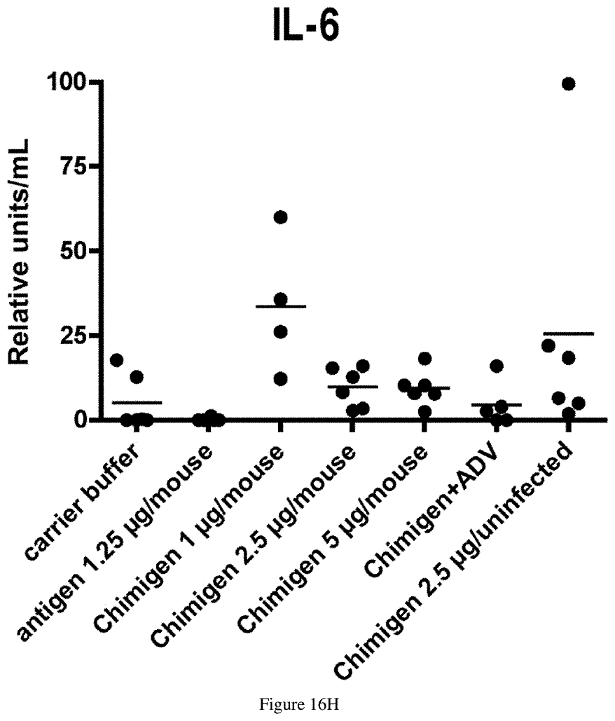

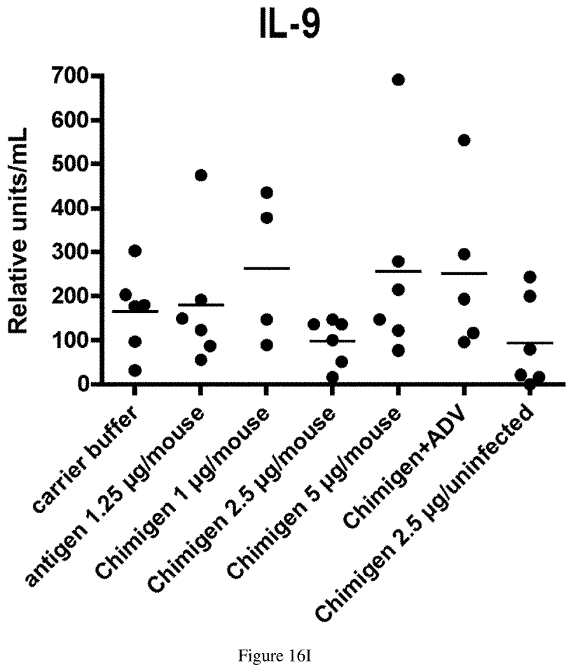

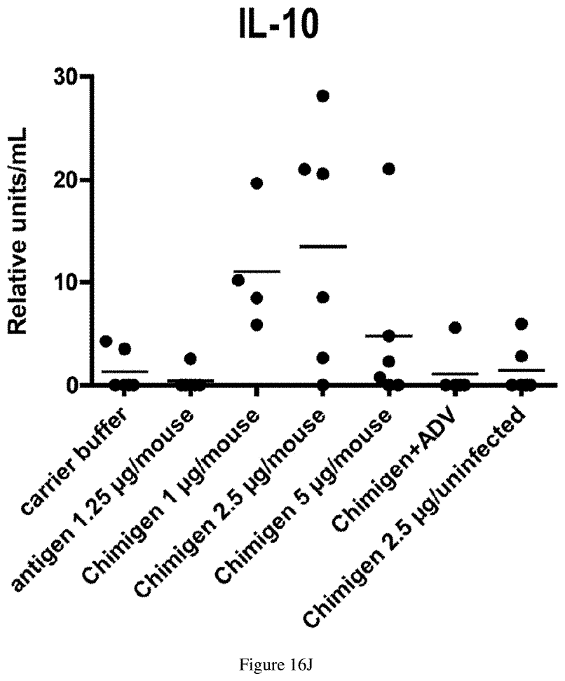

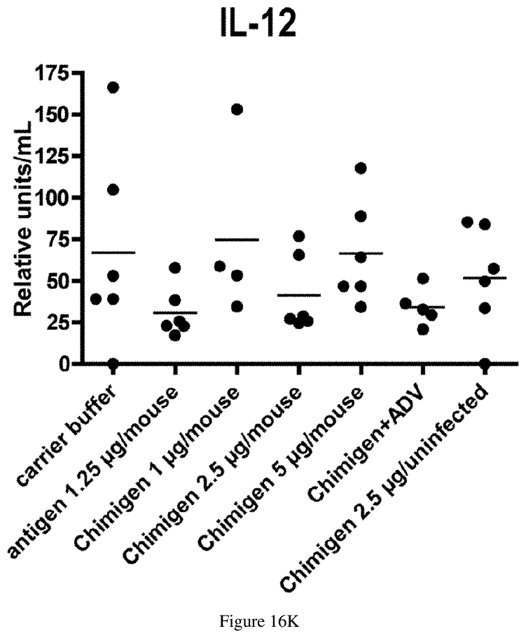

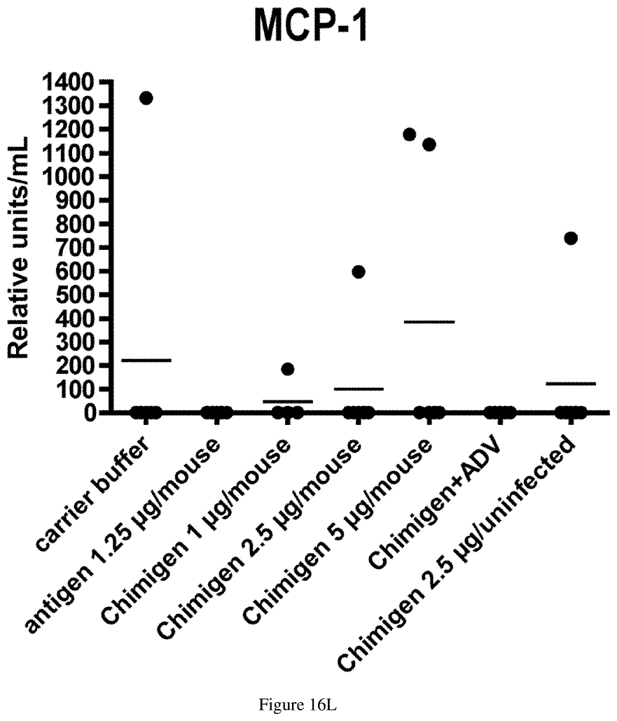

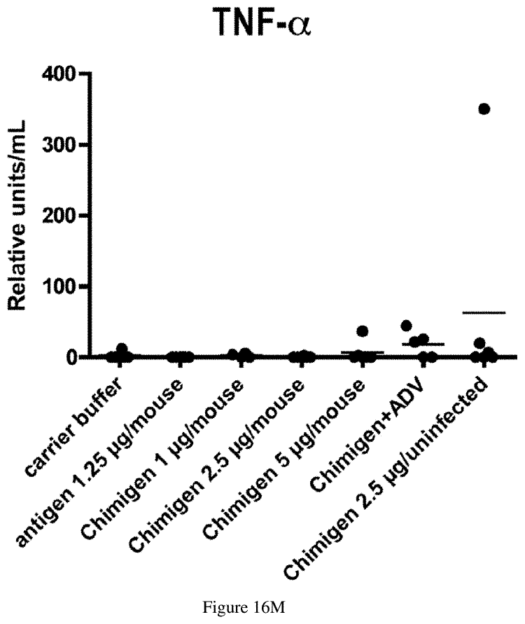

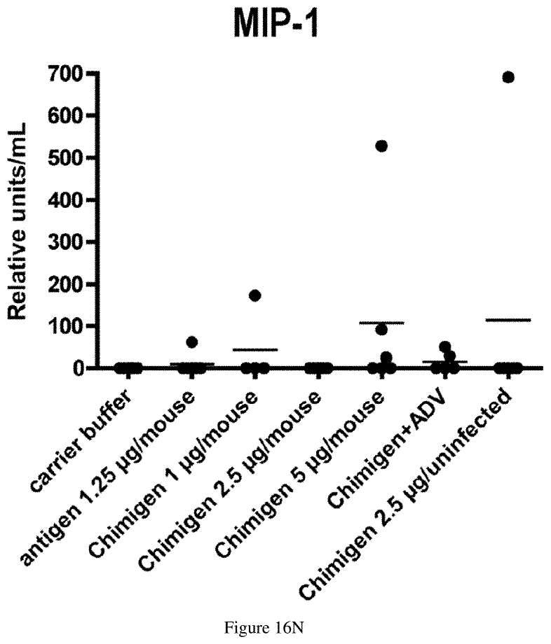

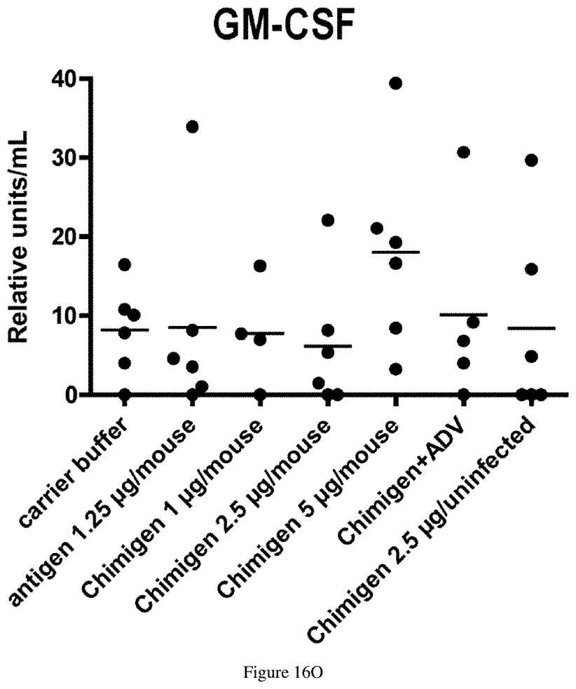

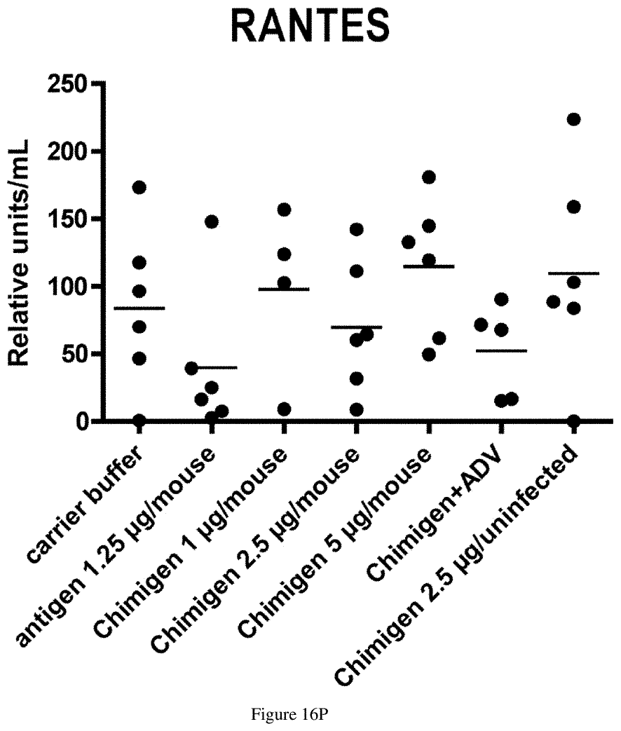

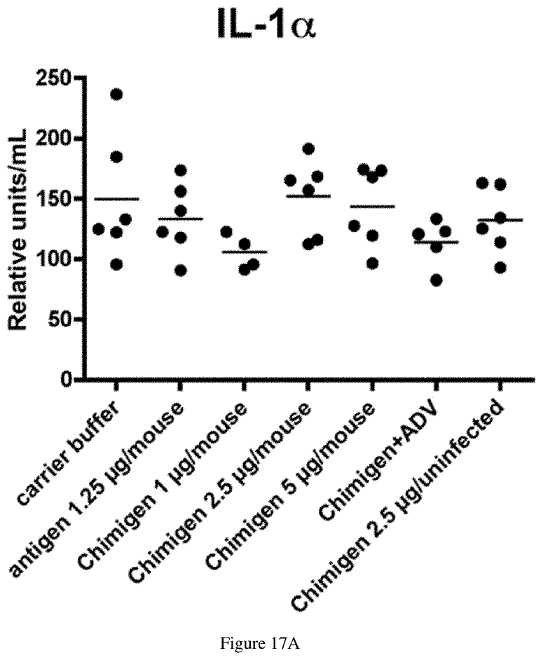

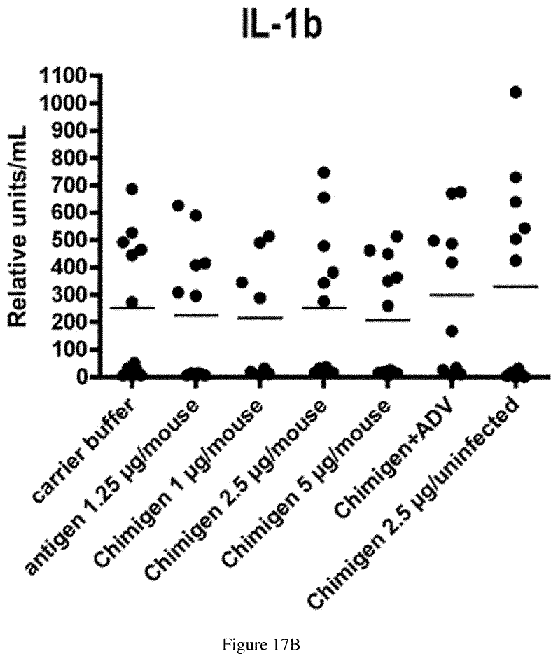

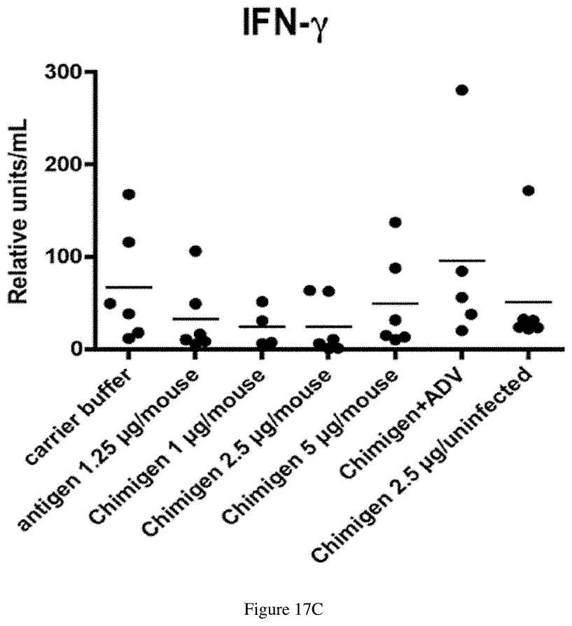

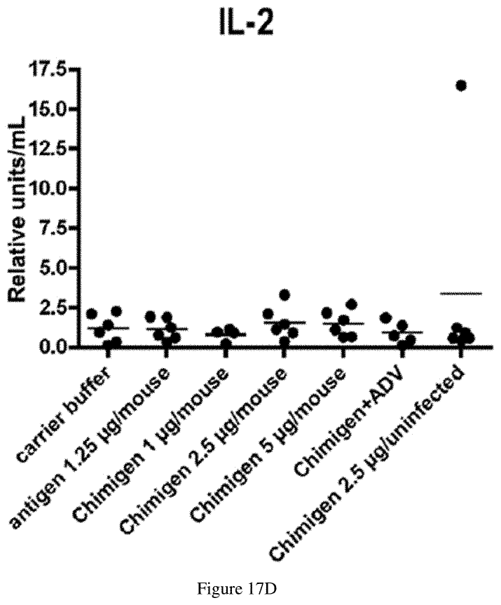

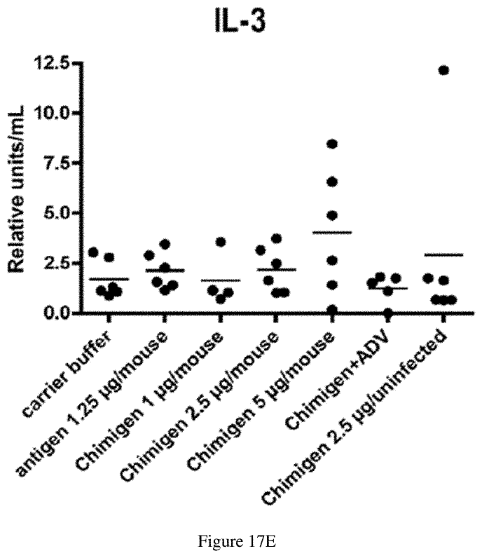

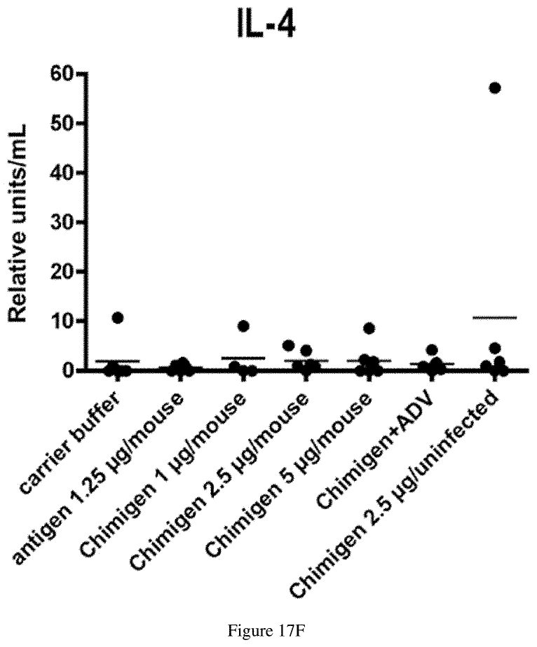

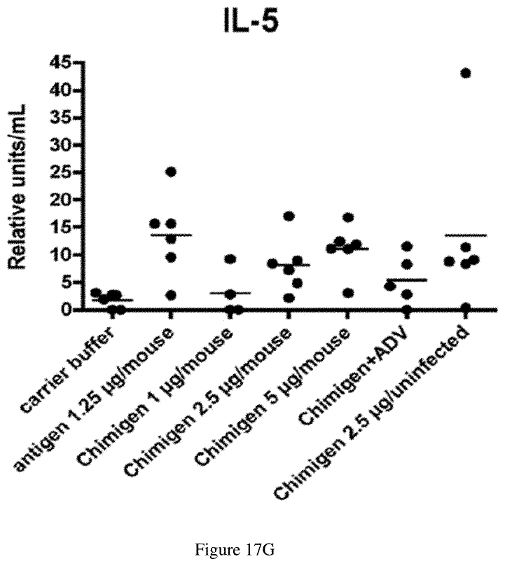

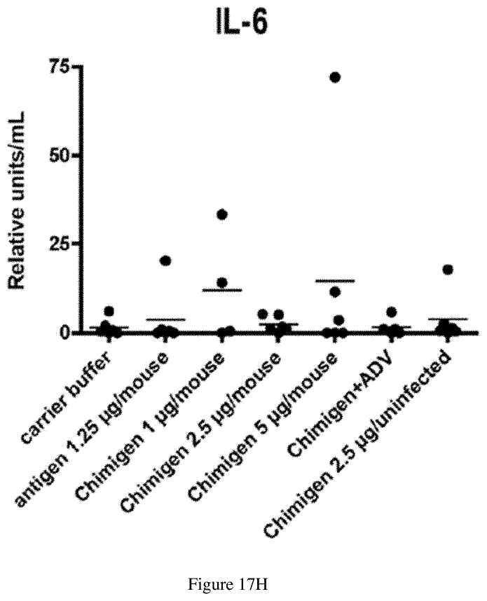

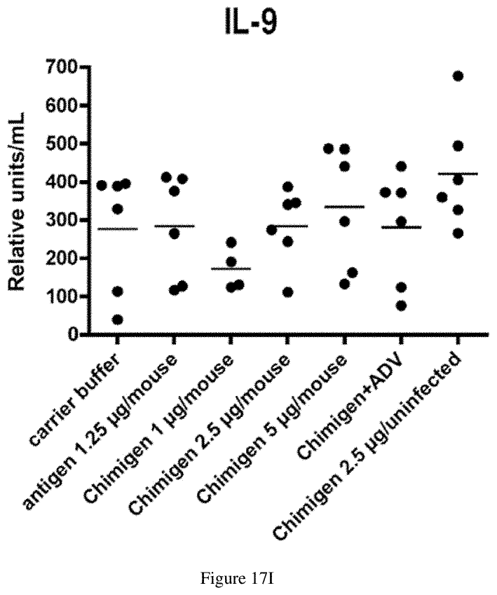

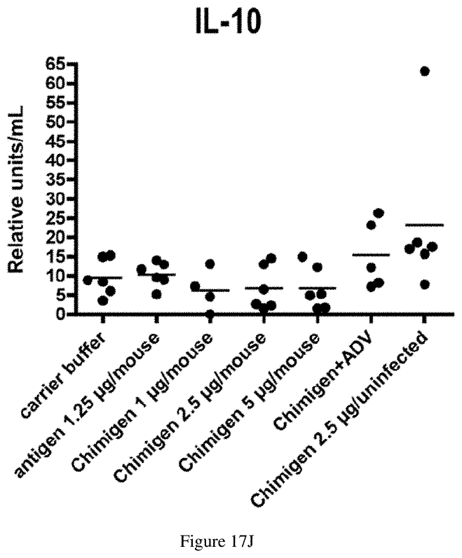

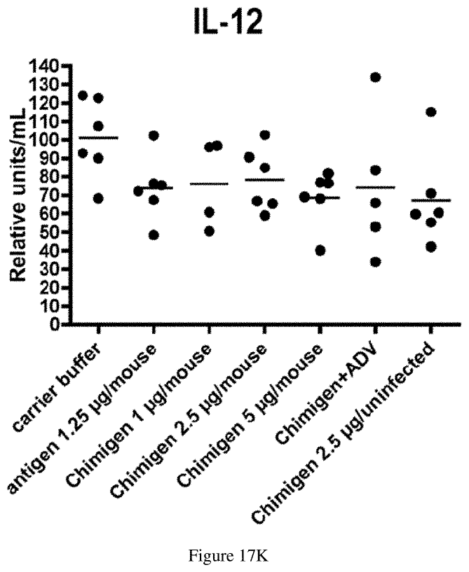

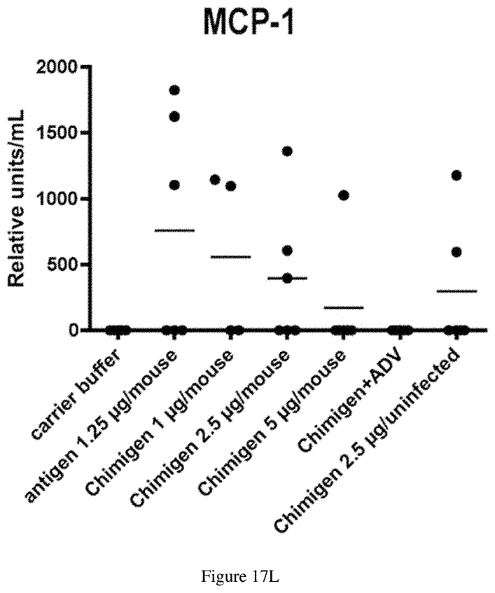

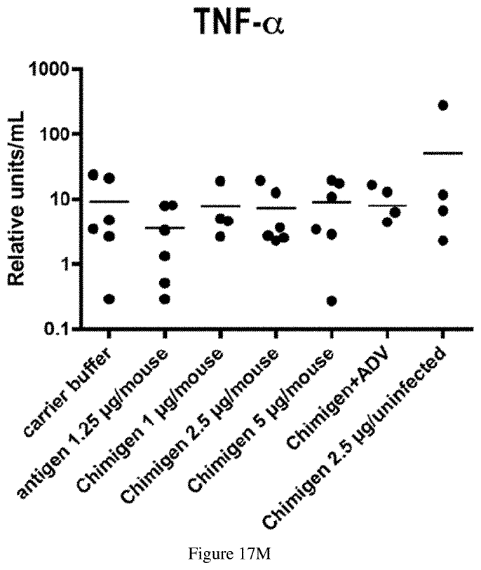

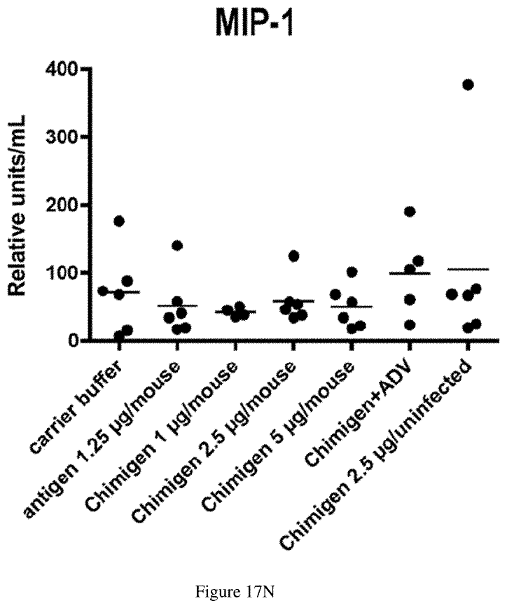

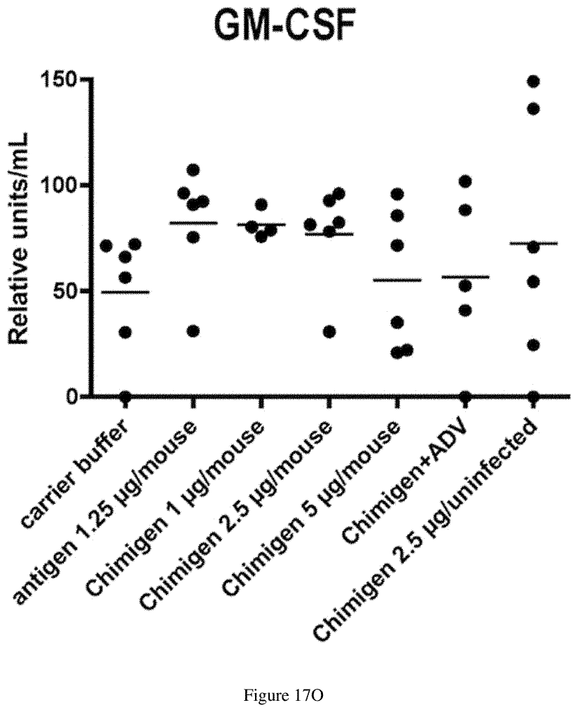

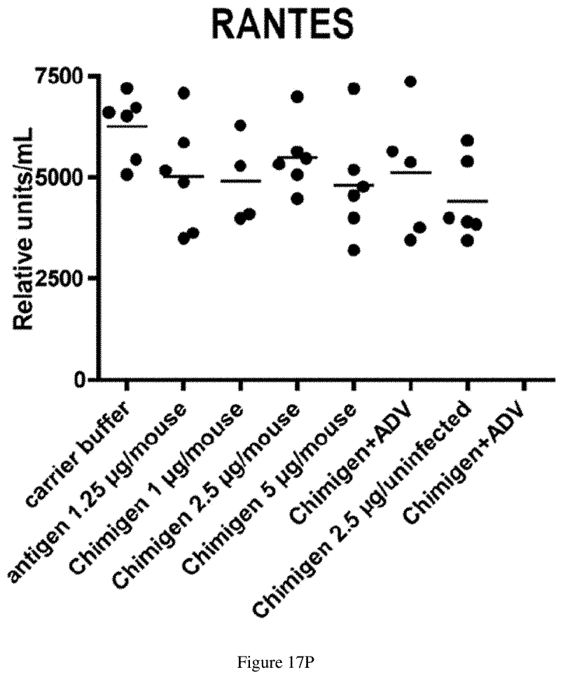

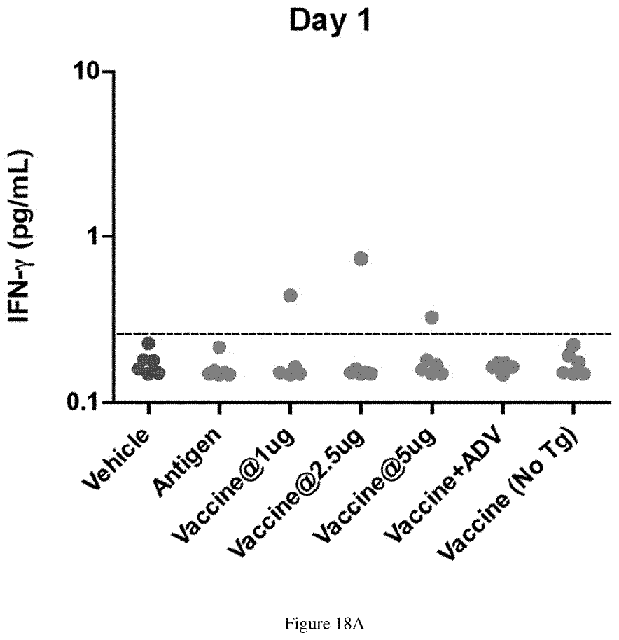

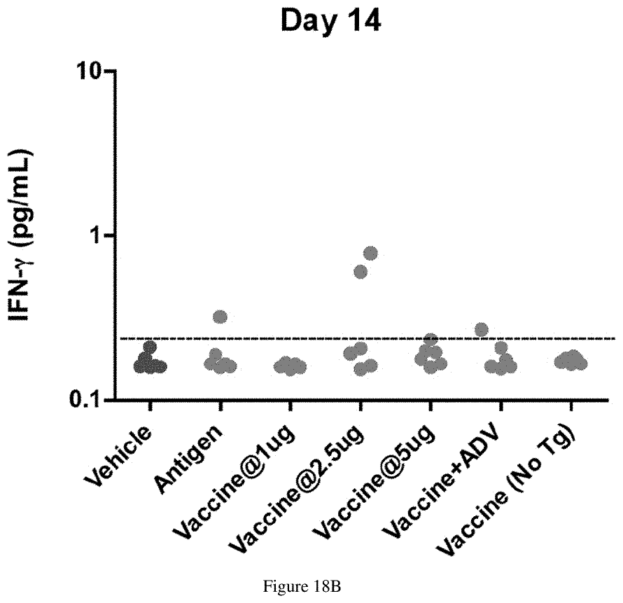

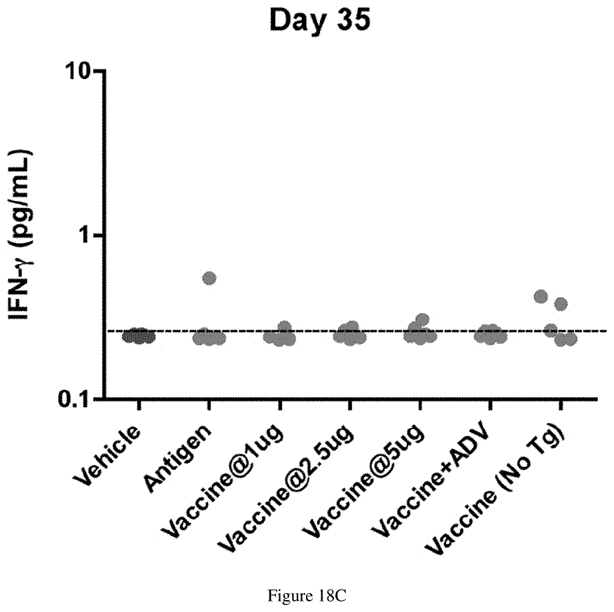

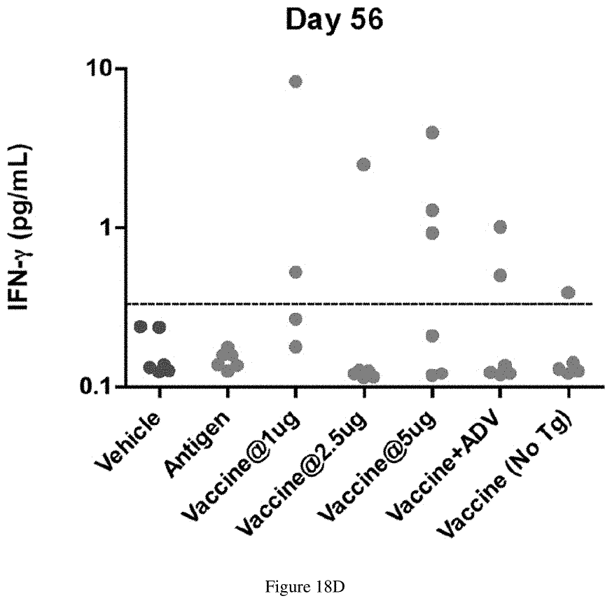

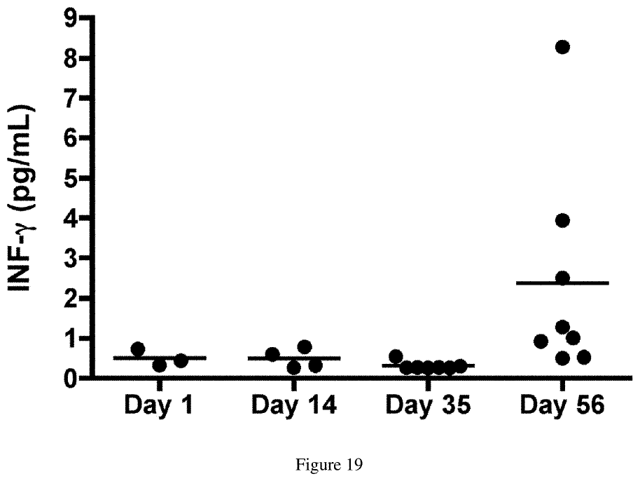

According to the Quansys™ arrays, HBV Core vaccine did not appreciably affect cytokine arrays, including IFN-γ, performed on liver (FIGS. 15A-15P), plasma (FIGS. 16A-16P), and spleen (FIGS. 17A-17P) tissues the day of necropsy. Since IFN-γ may be a marker for virus-specific cytotoxic T cells (CTL), using an ELISA kit specifically for mouse IFN-γ, we also assayed plasma collected 2 weeks after each vaccination, i.e., days 14, 35, and 56, with day 1 being a background control (FIGS. 18A-18D). The background (dashed lines) was calculated as three standard deviations of the mean so that samples above the background were considered positive for IFN-γ. Some samples from four mice collected before administration of the vaccine (day 1) were above the background levels (FIG. 18A). This was probably due to challenge with erroneous antigens from the environment and not to HBV-specific CTL responses. Similar numbers of four and three plasma samples from days 14 (FIG. 18B) and 35 (FIG. 18C), respectively, were also above the background, which suggests that this is due to erroneous elevated CTL responses. However, nine samples at day 56 (FIG. 18D) were above background levels, with six of the samples higher than any of the other IFN-γ values at the earlier time points. FIG. 19 shows the combined values from mice treated with any HBV Core vaccine treatment. Even though the groups were not statistically different, day-56 samples had noticeably higher IFN-γ values, which suggests that three injections of vaccine 2 weeks apart may have broken HBV-specific tolerance in some of the transgenic mice.

2.4 Conclusion

Three intradermal injections of vaccine 2 weeks apart may have broken HBV-specific tolerance in some of the transgenic mice as determined by elevation of plasma IFN-γ.

### REFERENCES

Banchereau, J., and Steinman, R. M. (1998). Dendritic cells and the control of immunity. Nature 392, 245-252.

Beasley, R. P. (1988). Hepatitis B virus. The major etiology of hepatocellular carcinoma. Cancer 61, 1942-1956.

Bouchard, M. J. and Schneider, R. J (2004). The enigmatic X gene of Hepatitis B virus. J. Viro1.78: 12725-12734.

Block, T. M., Rawat,S., and Brosgart, C. L (2015). Chronic hepatitis B: A wave of new therapies on the horizon. Antiviral Res. 121: 69-81.

Block, T. M., Alter, H., Brown, N et al. (2018). Research priorities fro the discovery of a cure for chronic hepatitis B: Report of a workshop. Antiviral Res. 150: 93-100.

Block, T. M., Alter, H. A., London, W. T and Bray, M. (2016). A historical perspective on the discovery and elucidation of the hepatitis B virus. Antiviral Res. 131: 109-123.

Bertoletti, A and Maini, M. K (2000). Protection or damage: A dual role for the virus-specific cytotoxic T lymphocyte response in hepatitris B and C infection? Curr. Opin. Microbiol. 3: 387-392.

Bertoletti, A., D'Elios, M. M., Boni, C., De Carli, M., Zignego, A. L., Durazzo, M., issale, G., Penna, A., Ficcadori, F., Del Prete, G and Ferrari, C. (1997). Different cytokine profiles of intrahepatic T cells in chronic hepatitis B and hepatitis C virus infections. Gastroenterology 112: 193-199.

Campton, K., Ding, W., Yan, Z., Ozawa, H., Seiffert, K., Miranda, E., Lonati, A., Beissert, S., and Granstein, R.D. (2000). Tumor antigen presentation by dermal antigen-presenting cells. J Invest Dermatol 115, 57-61.

Chang, S. F., Netter, H. J., Hildt, E., Schuster, R., Schaefer, S., Hsu, Y. C., Rang, A and Will, H. (2001). Duck hepatitis B virus expresses a regulatory HBx-like protein from a hidden open reading frame. J. Virol. 75: 161-170.

Chisari, F. V and Ferrari, C (1995). Hepatitis B virus immunopathogenesis. Annu. Rev. Immunol. 13: 29-60.

Donnelly, J. J., Ulmer, J. B., Shiver, J. W., and Liu, M. A. (1997). DNA vaccines. Annu Rev Immunol 15, 617-648.

Fisicaro, P., Valdatta,C., Massari, M., Loggi, E and Biasini, E (2010). Antiviral intrahepatic T-cell responses can be restored by blocking programmed death-1 pathway in chronic hepatitis B. Gastroenterol. 138: 682-693.

Fong, L., and Engleman, E. G. (2000). Dendritic cells in cancer immunotherapy. Annu Rev Immunol 18, 245-273.

Ganem, D (2001). The X files. One step closer to closure. Science 294: 2376-2378.

Geijtenbeek, T. B. H., van Vliet, S. J., Engering, A., Hart, B. A., and van Kooyk, Y. (2004). Self- and nonself-recognition by C-Type lectins on dendritic cells. Ann. Rev. Immunol. 22: 33-54.

George, R., Ma, A., Motyka, B., Shi, Y. E., Liu, Q. and Griebel, P. (2020). A dendritic cell-targeted chimeric hepatitis B virus immunotherapeutic vaccine induces both cellular and humoral immune responses in vivo. Hum Vaccin Immunother. 16(4):779-792.

George, R., Motyka, B., Ma, A., Wang, D., Eng, H., Xu, B., Polakowski, R., Noujaim, A. A., and Tyrrell, D. L. J. (2005). A new class of therapeutic vaccines produced in insect cells for the treatment of chronic viral infections. BioProcessing J. 4: 39-45.

George, R., Tyrrell, D. L. J., Noujaim, A. A., Ma, A., Xu, B., Wang, D., Eng, H., Polakowski, R., Szpacenko, A., and Motyka, B. (2006). A new class of therapeutic vaccines for the treatment of chronic hepatitis B infections. In “Framing the Knowledge of Viral Hepatitis” Schinazi, R. F and Schiff, E. R Editors, IHL Press USA.

Guidotti, L. G., Borrow, P., Hobbs, M., Matzke, B., Gresser, I., Oldstone, M. B and Chisari, F. V. (1996a). Viral cross talk: Intracellular inactivation of the hepatitis B virus during an unrelated viral infection of the liver. Proc. Natl. Acad. Sci. USA 93: 4589-4594.

Guidotti, L. G., Ishikawa, T., Hobbs, M., Matzke, B., Schreiber, R and Chisari, F. V. (1996). Intracellular inactivation of the hepatitis B virus by cytotoxic T lymphocytes. Immunity 4: 25-36.

Guidotti, L. G., Matzke, B., Schaller, H., and Chisari, F. V. (1995). High-level hepatitis B virus replication in transgenic mice. Journal of Virology 69, 6158-6169.

Guidotti, L. G., Rochford, R., Chung, J., Shapiro, M., Purcell, R and Chisari, F. V. (1999). Virus clearence without destruction of infected cells during acute HBV infection. Science 284: 825-829.

Gust, I. D., Burrell, C. J., Robinson, W. S and Zuckerman, A. J (1986). Taxonomic classification of human hepatitis B. Intervirology 25: 14-29.

Hafkemeyer, P., Keppler-Hafkemeyer, A., al Haya, M. A., von Janta-Lipinski, M., Lehmann, C., Offensperger, W. B., Offensperger, S., Gerok, W and Blum, H. E (1996). Inhibition of duck hepatitis B virus replication by 2′,3′-dideoxy-3′-fluoroguanosine in vitro and in vivo. Antimicrob. Agents Chemotherap. 40: 792-794.

Heuss,L., Heim, M. H., Schultz, U., Wissmann, D., Offensperger, W. B., Staeheli, P and Blum H. E. (1988). Biological efficacy and signal transduction through STAT proteins of recombinant duck interferon in duck hepatitis B virus infection. J. Gen. Virol. 79: 2007-2012.

Hilgers, L. A., Lejeune, G., Nicolas, I., Fochesato, M., and Boon, B. (1999). Sulfolipo-cyclodextrin in squalane-in-water as a novel and safe vaccine adjuvant. Vaccine 17, 219-228.

Jilbert, A. R., Botten, J. A., Miller, D. S., Betram, E. M., Hall, P., Kotlarski, I and Burrell, C. J. (1998). Characterization of age- and dose-related outcomes of duck hepatitis B virus infection. Virology 243: 273-282.

Jilbert, A. R and Kotlarski, I. (2000). Immune response to duck hepatitis B virus infection. Dev. Comp. Immunol. 24: 285-302.

Klein, N. P and Schneider, R. J (1997). Activation of Src family kinases by Hepatitis B virus HBx protein and coupled signaling to Ras. Mol. Cell. Biol. 17: 6427-6436.

Lai, W. C., and Bennett, M. (1998). DNA vaccines. Crit Rev Immunol 18, 449-484. Larsson, M., Fonteneau, J. F., and Bhardwaj, N. (2001). Dendritic cells resurrect antigens from dead cells. Trends Immunol 22, 141-148.

Laupeze, B., Fardel, O., Onno, M., Bertho, N., Drenou, B., Fauchet, R., and Amiot, L. (1999). Differential expression of major histocompatibility complex class Ia, Ib, and II molecules on monocytes-derived dendritic and macrophagic cells. Hum Immunol 60, 591-597.

Lau, J. Y. and Wrught, T. L. (1993). Molecular virology and pathogenesisi of hepatitis B. Lancet 342: 1335-1340

Le Guerhier, F., Thermet, A., Guerret, S., Chevallier, M., Jamard, C., Gibbs, C. S., Trepo, C., Cova, L and Zoulim, F (2003). Antiviral effect of adefovir in combination with a DNA vaccine in the duck hepatitis B infection model. J. Hepatol. 38: 328-334.

Liang, T. J., Block, T. M, McMahon, B. J., Ghany, M. G., Urban, S., Guo, J. T., Locarini, S., Zoulim, F., Chang, K. M. m and Lok, A. S. (2015). Present and future therapies of hepatitisB: from discovery to cure. Heatology 62:1893-1908

Lorenz, M. G., Kantor, J. A., Schlom, J., and Hodge, J. W. (1999). Anti-tumor immunity elicited by a recombinant vaccinia virus expressing CD70 (CD27L). Hum Gene Ther 10, 1095-1103.

Ma, A., Motyka, B., Gutfreund, K., Shi, Y. E. and George, R. (2020). A dendritic cell receptor-targeted chimeric immunotherapeutic protein (C-HBV) for the treatment of chronic hepatitis B. Hum Vaccin Immunother. 16(4):756-778.

Mason, W. S., Seal, G., and Summers, J. (1980). Virus of Pekin ducks with structural and biological relatedness to human hepatitis B virus. Journal of virology 36, 829-836.

Newman, K. D., Elamanchili, P., Kwon, G. S., and Samuel, J. (2002). Uptake of poly(D,L-lactic-co-glycolic acid) microspheres by antigen-presenting cells in vivo. J Biomed Mater Res 60, 480-486.

Marion, P. L., Knight, S. S., Ho, B. K., Guo, Y. Y.,Robinson, W. S. and Popper, H. (1984). Liver disease associated with duck hepatitis B virus infection of domestic ducks. Proc Natl Acad Sci USA 81: 898-902

Marion, P. L., Oshiro, L. S., Regnery, D. C., Scullard, G. H., and Robinson, W. S. (1980). A virus in Beechey ground squirrels that is related to hepatitis B virus of man. Proc. Natl. Acad. Sci. USA 77: 2941-2945.

Mason, W. S., Seal, G and Summers, J. (1980). Virus of Pekin ducks with structural and biological relatedness to human hepatitis B virus. J. Virol. 36: 829-836.

Meier, P., Scougall, C. A., Will, H., Burrell, C. J and Jilbert, A. R. (2003). A duck hepatitis B virus strain with a knockpout mutation in the putatitve X ORF shows similar infectivity and in vivo growth characteristics to wild-type virus. Virology 317: 291-298.

Newman, K. D., Kwon, G. S., Miller, G. G., Chlumecky, V., and Samuel, J. (2000). Cytoplasmic delivery of a macromolecular fluorescent probe by poly(d, 1-lactic-co-glycolic acid) microspheres. J Biomed Mater Res 50, 591-597.

Newman, K. D., Samuel, J., and Kwon, G. (1998). Ovalbumin peptide encapsulated in poly(d,l lactic-co-glycolic acid) microspheres is capable of inducing a T helper type 1 immune response. J Control Release 54, 49-59.

Peng, G., Li, S., Wu, W., Tan, X., Chen, Y and Chen, Z (2008). PD-1 upregulation is associated with HBV-specific T cell dysfunction in chronic hepatitis B patients. Mol. Immunol. 45: 963-970.

Regnault, A., Lankar, D., Lacabanne, V., Rodriguez, A., Thery, C., Rescigno, M., Saito, T., Verbeek, S., Bonnerot, C., Ricciardi-Castagnoli, P., and Amigorena, S. (1999). Fcgamma receptor-mediated induction of dendritic cell maturation and major histocompatibility complex class I-restricted antigen presentation after immune complex internalization. J Exp Med 189, 371-380.

Offensperger, W. B., Offensperger, S., Keppler-Hafkemeyer, A., Hafkemeyer, P and Blum H. E (1996). Antiviral activities of pencyclovir and famcyclovir on duck hepatitis B virus in vitro and in vivo. Antivir. Ther. 1: 141-146.

Offensperger, W. B., Offensperger, S., Walter, E., Teubner, K., Igloi, G., Blum, H. E and Gerok, W. (1993). In vivo inhibition of duck hepatitis B virus replication and gene expression by phosphorothiolate modified antisense oligonucleotides. EMBO J. 12: 1257-1262.

Rehermann, B (2000). Intrahepatic T cells in hepatitis B: Viral control versus liver cell injury. J. Exp. Med. 191: 1263-1268.

Rollier, C., Charollois, C., Jamarad, C., Trepo, C and Cova, L (2000a). Early life humoral response of ducks to DNA immunization against hepadnavirus large envelope protein. Vaccine 18: 3091-3096.

Rollier, C., Charollois, C., Jamarad, C., Trepo, C and Cova, L (2000b). Maternally tranferred antibodies from DNA-immunized avians protect offspring against hepadnavirus infection. J. Virol. 74: 4908-4911.

Rollier, C., Sunyach, C., Barraud, L., Madani, N., Jamarad, C., Trepo, C and Cova, L (1999).Protective and therapeutic effect of DNA-based immunization against hepadnaviral large envelope protein. Gastroenterology 116: 658-665.

Schultz, U and Chisari, F. V. (1999). Recombinant duck interferon gamma inhibits duck hepatitis B virus replication in primary hepatocytes. J. Virol. 73: 3162-3168.

Schultz, U., Grgacic, E and Nassal, M (2004). Duck hepatitis B virus: an invaluable model system for HBV infection. Adv. Virus Res. 63: 1-70.

Sharma, J. M (1997). The structure and function of the avian immune system. Acta Vet. Hung. 45: 229-238.

Sondermann, P., Kaiser, J and Jacob, U (2001). Molecular basis for immune complex recognition: A comparison of Fc-receptor structures. J. Mol. Biol. 301: 737-749.

Sprengel, R., Kaleta, E. F., and Will, H. (1988). Isolation and characterization of a hepatitis B virus endemic in herons. Journal of virology 62, 3832-3839.

Steinman, R. M., Inaba, K., Turley, S., Pierre, P., and Mellman, I. (1999). Antigen capture, processing, and presentation by dendritic cells: recent cell biological studies. Hum Immunol 60, 562-567.

Summers, J., Smolec, J. M., and Snyder, R. (1978). A virus similar to human hepatitis B virus associated with hepatitis and hepatoma in woodchucks. Proceedings of the National Academy of Sciences of the United States of America 75, 4533-4537.

Tarte, K., and Klein, B. (1999). Dendritic cell-based vaccine: a promising approach for cancer immunotherapy. Leukemia 13, 653-663.

Tzeng, H. T., Tsai, H. F., Liao, H. J., Lin, Y. J., Chen, L., Chen, P. J.and Hsu, P. N. (2012). PD-1 blockade reverses immune dysfunction and hepatitis B viral persistence in a mouse animal model. PLoS One 7: e39178

Urban, M. K., O'Connell, A. P and London, W. T. (1985). Sequence of events in natural infection of Pekin duck embryos with duck hepatitis B virus. J. Virol. 55:16-22.

Vickery, K and Cosssart, Y. E (1996) DHBV manipulation and prediction of the outcome of infection. J. Hepatol. 25: 504-509.

Wen, Y. M., Qu, D., and Zhou, S. H. (1999). Antigen-antibody complex as therapeutic vaccine for viral hepatitis B. Int Rev Immunol 18, 251-258.Zinkernagel, R. M. (1996). Immunology taught by viruses. Science 271: 173-178.

Zinkernagel, R. M. (1996). Immunology taught by viruses. Science 271: 173-178.

What is claimed is:

1. A method of treating viral infection in a host comprising administering a chimeric antigen and a therapeutic agent that decreases viral levels in the host.

2. The method of claim 1, wherein the chimeric antigen is HBV S1/S2/Core chimeric antigen.

3. The method of claim 2, wherein the therapeutic agent is an HBV antiviral agent.

4. The method of claim 3, wherein the HBV antiviral agent is a pharmaceutically acceptable agent comprising one or more compounds selected from the group consisting of: tenofovir, tenofovir disoproxil fumarate, tenofovir alafenamide, entecavir, telbuvidine, adefovir dipivoxil, and lamivudine.

5. The method of claim 3, wherein the HBV antiviral agent is an HBV antiviral RNAi agent that inhibits HBV replication; or the HBV antiviral agent is a core capsid assembly inhibitor, or a TLR agonist.

6. The method of claim 1, further comprising administering an immune modulatory agent to the host.

7. The method of claim 6, wherein the immune modulatory agent is an interferon such as Peginterferon α2-a.

8. The method of claim 6, wherein the immune modulatory agent is an immune checkpoint inhibitor such as PD1/PDL1 inhibitor.

9. The method of claim 1, wherein the host is a human subject.

10. The method of claim 1, wherein the viral infection is an HBV infection.

11. A kit for treating viral infection in a host, comprising a chimeric antigen and a therapeutic agent that decreases viral levels in the host, and a package insert or label with directions to treat viral infection in the host by administering to the host the therapeutic chimeric antigen and a therapeutic agent.

12. The kit of claim 11, wherein the directions comprise instructions to administer the chimeric agent and the therapeutics agent simultaneously.

13. The kit of claim 11, wherein the directions comprise instructions to administer the chimeric agent and the therapeutic agent separately.

14. The kit of claim 11, wherein the chimeric antigen is HBV S1/S2/Core chimeric antigen.

15. The kit of claim 11, wherein the therapeutic agent is an HBV antiviral agent.

16. The kit of claim 15, wherein the HBV antiviral agent is a pharmaceutically acceptable agent comprising one or more compounds selected from the group consisting of: tenofovir, tenofovir disoproxil fumarate, tenofovir alafenamide, entecavir, telbuvidine, adefovir dipivoxil, and lamivudine.

17. The kit of claim 15, wherein the HBV antiviral agent is an HBV antiviral RNAi agent that inhibits HBV replication; or the HBV antiviral agent is a core capsid assembly inhibitor, or a TLR agonist.

18. The kit of claim 11, further comprising an immune modulatory agent.

19. The kit of claim 18, wherein the immune modulatory agent is an interferon such as Peginterferon α2-a.

20. The kit of claim 18, wherein the immune modulatory agent is an immune checkpoint inhibitor such as PD1/PDL1 inhibitor.


SEQUENCE LISTING (13 sequences)

SEQ ID NO: 1 - DNA, 55 bp, Artificial Sequence
  Synthetic Construct
tgtcattctgcggccgcaaggcggcggatccgtggacaagaaaattgtgcccagg

SEQ ID NO: 2 - DNA, 36 bp, Artificial Sequence
  Synthetic Construct
acgaatcaagctttgcagcccaggagagtgggagag

SEQ ID NO: 3 - DNA, 33 bp, Artificial Sequence
  Synthetic Construct
tgcgctaccatggatatcaatgcttctagagcc

SEQ ID NO: 4 - DNA, 43 bp, Artificial Sequence
  Synthetic Construct
tgtcattctgcggccgcgatttcctaggcgagggagatctatg

SEQ ID NO: 5 - DNA, 21 bp, Artificial Sequence
  Synthetic Construct
ataaaacgccgcagacacatc

SEQ ID NO: 6 - DNA, 22 bp, Artificial Sequence
  Synthetic Construct
aacctccaatcactcaccaacc

SEQ ID NO: 7 - DNA, 28 bp, Artificial Sequence
  Synthetic Construct
agcgataaccaggacaagttggaggaca

SEQ ID NO: 8 - DNA, 21 bp, Artificial Sequence
  Synthetic Construct
ggacaaacgggcaacatacct

SEQ ID NO: 9 - DNA, 23 bp, Artificial Sequence
  Synthetic Construct
tcttcctcttcatcctgcctgct

SEQ ID NO: 10 - DNA, 30 bp, Artificial Sequence
  Synthetic Construct
tccagaaagaaccaacaagaagatgaggca

SEQ ID NO: 11 - DNA, 21 bp, Artificial Sequence
  Synthetic Construct
gcatcttgggctacactgagg

SEQ ID NO: 12 - DNA, 22 bp, Artificial Sequence
  Synthetic Construct
gaaggtggaagagtgggagttg

SEQ ID NO: 13 - DNA, 28 bp, Artificial Sequence
  Synthetic Construct
accaggttgtctcctgcgacttcaacag